(19) United States
(12) Patent Application Publication
(10) Pub. No.: US 2026/0256384 A1
(43) Pub. Date: Sep. 3, 2026

(54) AN INTEGRATED DIGIT IN NOISE TEST TO EVALUATE HEARING AND COGNITIVE FUNCTION

(71) Applicant: The University of Hong Kong, Hong Kong (HK)

(72) Inventors: Lai Nar Lena WONG, Hong Kong (HK); Shangqiguo WANG, Hong Kong (HK); Jaden Yuen Ho TAO, Gordon (AU)

(21) Appl. No.: 19/165,158

(22) PCT Filed: Mar. 14, 2024
(86) PCT No.: PCT/CN2024/081654

§ 371 Date: Sep. 15, 2025

Related U.S. Application Data
(60) Provisional application No. 63/490,032, filed on Mar. 14, 2023.
(60) Provisional application No. 63/514,903, filed on Jul. 21, 2023.

(30) Foreign Application Priority Data
Dec. 5, 2023 (HK) 32023083669.6

(51) Int. Cl. A61B 5/12; A61B 5/00

(52) U.S. Cl. CPC A61B 5/123; A61B 5/4088; A61B 5/4803; A61B 5/7271; A61B 5/741; A61B 5/742; A61B 5/7475; A61B 2560/0242

(57) ABSTRACT

A testing method of testing a participant's ability to detect or understand speech, and evaluate hearing, speech understanding and processing, and cognitive function. The method includes: (a) audibly presenting a series of 2 digits, 3 digits, 4 digits, 5 digits, in random or fixed orders, to both ears or a first ear of a participant in quiet or in noise at a fixed or varied. The digits can be in any language; (b) repeating the same procedures as (a) to a second ear (c) recording digits or digit sequences identified correctly by the participant; and (d) recoding reaction time to repeat digits, (e) calculating scores based on the percentage of digits or digit sequences repeated correctly by the participant or the difference in level between speech and noise to yield 50% of the digit sequences being identified correctly.

### FIELD OF THE INVENTION

The invention is in the field of hearing and cognitive function, for example, in geriatric healthcare.

### BACKGROUND OF THE INVENTION

Assessing outcomes with hearing devices is important in ensuring optimal benefit. Although there is a number of tests available for assessing speech understanding (e.g., the Hearing-in-Noise Test (HINT) and the Matrix Sentence Test), they have not been widely adopted for several reasons. First, people with severe hearing loss may have difficulty hearing the sentences even with hearing devices. Second, older adults with cognitive decline may find it difficult to repeat sentences. Finally, individuals with low education may not perform as well on these tests. Thus, a hearing function test that employs simple test stimuli such as digits would facilitate the evaluation of older adults. A test that evaluates digit recognition in noise, such as Digit-in-Noise Tests (DINTs), would emulate listening environment that individuals with hearing loss find most challenging.

As all Digit-in-Noise Tests (DINTs) use digit triplets as test stimuli, they are also referred to as Digit Triplet Tests (DTTs). Since the development of the Dutch version in 2004, the DTT is now available in over 20 languages (Smits et al., 2004). While measurement errors of less than 1 dB have been found for test-retest reliability in most language versions of the DTT, speech recognition thresholds (SRT) have also been found to correlate well with four frequency-average pure-tone thresholds (Van den Borre et al., 2021). With an appropriate cut-off point, the DTT can effectively differentiate between listeners with normal and impaired hearing with high test sensitivity and specificity (Smits et al., 2004). Smits et al. (2013) reported that SRTs obtained with the new version of the Dutch DTT correlated highly (r=0.96) with SRTs measured using the Dutch sentences-in-noise test among listeners with normal hearing (NH) and various degrees of simulated hearing loss, suggesting good criterion validity. As no learning effect was observed after one practice list, and the DTT can be administrated and completed independently by the users, the test can be administered within a short time. The DTT has been used successfully in hearing screening in countries such as the Netherlands and South Africa (Akeroyd et al., 2015; Kwak et al., 2022; Potgieter et al., 2018; Smits et al., 2004, 2013; Van den Borre et al., 2021). In recent years, the DTT has also been used for evaluating hearing device outcomes (Cullington & Aidi, 2017; Kaandorp et al., 2015). Age-related hearing loss is associated with the deterioration of sensory hair cells in the inner ear. While hearing devices can make sounds louder and easier to perceive, they cannot fully correct the distortion caused by these damaged hair cells. Thus, it is important to evaluate the speech understanding ability of hearing aid users.

While digit triplet recognition in noise relies primarily on bottom-up auditory processing, which does not involve much linguistic skill. (Smits et al., 2013), listening to digits in noise theoretically triggers explicit processing involving auditory memory and other cognitive resources. In other words, the ability to remember a digit sequence is tied to auditory memory (such as short-term memory and working memory), which is often reduced in older adults, particularly those with hearing loss (Nyberg et al., 2012). According to the Ease of Language Understanding (ELU) model (Rönnberg et al., 2013, 2019, 2021), when listening in challenging conditions, such as when the listener is suffering from hearing loss or listening in noise, distorted speech information cannot be matched with the phonological representations in semantic long-term memory. Top-down explicit remedial processing, which is highly dependent on cognitive function such as working memory, is then required to remedy the mismatch for speech understanding, whereby reduced working memory could result in poorer speech discrimination (Akeroyd, 2008). As the number of digits increases (working memory load increases), the required cognitive resources for speech recognition should increase accordingly. Consequently, the number of digits would have an impact on speech recognition in noise, particularly when the listener is experiencing cognitive decline.

Although various language versions of the DINT use digit triplets as test stimuli, little research has examined the effects of the number of digits. Smits et al. (2004) argued that digit triplets yield better test efficiency than digit pairs, and that speech perception in noise using more than three digits probably places a greater demand on cognitive memory capacity (Smits et al., 2004). While Wilson et al. (2005) reported no appreciable difference in the recognition of digit pairs (2-digit) versus triplets in babble noise by listeners with normal and impaired hearing, they recommended digit pairs for clinical use due to shorter test time and reduced demand on memory (Wilson et al., 2005). Wang and Wong (2023) found a slight increase in speech level is needed to recognize longer digit sequences, such as 5-digits, compared to 2-digit and 3-digit sequences, among those with normal cognitive function. Tripathi et al. (2019) also reported that adults aged 50 to 80 in India were able to handle more than five digits in a forward Digit Span Test (DST) (Tripathi et al., 2019).

Research in our lab, however, shows that those with cognitive decline are not able to handle longer digit sequences, compared to 2-digit and 3-digit sequences, when listening in noise. Cognitive decline in older adults leads to a decreased ability to allocate cognitive resources effectively. This means they struggle to fill in missing information due to hearing loss or when background noise interferes with speech signals.

Another problem in speech perception tests involves the multilingual nature of our world. Because of this multilingual aspect, a language and dialect specific test is needed for each population, given that dialect and sometimes “minor” differences such as accent could change performance on a test. Because scientific expertise and knowledge in the psychometrics of test development are required to develop and validate tests, currently available tests are available only in 20 or so commonly spoken languages such as American English, British English, or where healthcare associated with auditory issues are well developed (e.g., Danish, Swedish, German). Clinicians working with other populations have to depend on clinical judgement and self-reports from patients to estimate whether there is a speech comprehension problem. Even so, it is challenging for them to know the extent of the problem; thereby introducing an additional problem in assessment of hearing difficulties.

Consequently, older adults often experience difficulty in comprehending speech and may express frustration about the perceived limited benefits of hearing devices. In response, hearing health providers, such as audiologists and hearing aid dispensers, must develop effective management strategies. To evaluate the challenges in speech understanding, researchers and hearing healthcare providers traditionally use prerecorded test materials from a single speaker in sound-treated booths, using noise simulations that aim to mimic real-world environments.

However, research has demonstrated that the measured performance in controlled settings does not adequately represent the real difficulties faced in daily life scenarios. In everyday situations, the sources of noise change dynamically, and the acoustic environments are highly variable. For example, when listening on a street, traffic noise fluctuates with the distance, speed, and size of vehicles. In a restaurant, people's conversations mingle with the clatter of dishes. Factors such as room size, the number of talkers, the presence of different types of noise, reverberation, the talker's speech intensity and speed, the distance from the talker, the conversation's content, and other variables are virtually impossible to account for.

Therefore, evaluations conducted in sound booths can, at best, provide an estimate of the difficulties an individual may face in understanding speech. Clinicians are then tasked with counseling patients and adjusting hearing devices based on these rough estimates and imperfect representations of the noise environments encountered by patients. These challenges could result in hearing device owners not using their devices, leading to negative reviews that dissuade many others from considering hearing devices. For example, it has been estimated that in mainland China, only 10% of those who require hearing devices actually own them, and even fewer use them consistently.

Given recent research linking hearing loss to cognitive decline, social isolation, depression, falls, and other health conditions, there is an urgent need to help older individuals and their families better comprehend their hearing abilities and pursue suitable interventions. This also necessitates a deeper understanding of their hearing requirements by professionals working with them to optimize hearing device fitting parameters for enhanced speech comprehension. Accordingly, there is a need for improvement in a hearing loss assistance program. However, healthcare providers are often challenged by the fact that in the presence of a hearing loss in addition to cognitive impairment, the patient or older adult may not be able to hear the instructions of a cognitive function test well enough to yield an accurate assessment. While there are versions of these tests that would account for the presence of hearing loss, no test in the World examines the combined effects of a hearing loss and cognitive impairment, or separate the effects of one from the other on speech understanding ability.

The present invention aims to offer a speech test that is straightforward to administer and interpret, unaffected by the participant's literacy levels, enabling the differentiation between the impacts of cognitive decline and hearing loss. Additionally, it seeks to improve methods for assessing and developing test materials for a participant's speech detection capabilities, considering factors such as ethnicity, spoken language, and geographic location. Furthermore, an objective is to provide an apparatus and method for measuring digit recognition in relation to hearing loss in any test environment, thereby addressing the limitations found in previous approaches.

### SUMMARY OF THE INVENTION

A digit in noise test method that incorporates a few types of digit sequences as signals to evaluate speech understanding, is provided. The method employs up to 5 digit sequences in noise to evaluate hearing and cognitive function. By evaluating the ability to repeat shorter digit sequences in forward order (e.g., 2 or 3 digits) and longer digit sequences in forward order (e.g., 5 digits); comparing speech recognition using forward digit sequence versus backward digit sequence (e.g., repeating 3 digit sequence in forward vs backward orders); or comparing speech recognition using time compressed digits (e.g., forward 3 digits at 200 ms intervals versus 100 ms intervals); or evaluating speech recognition to digit sequences using other arrangements (e.g., presenting different digits to the two ears and asking the listeners to integrate the digits, or requiring the listener to pay attention to certain digits) for the first time a method is provided which allows the end user to understand additional difficulties resulting from cognitive decline, if any; and differentiate the difficulties due to problems in hearing and cognition. The disclosed method eliminates the need to estimate or guess how results from separate measurements (i.e., hearing tests and cognitive function measurements) should be integrated.

The test can be conducted at a fixed signal-to-noise ratio (i.e., the difference in level between speech and noise) to obtain percent correct scores or as an adaptive procedure (signal-to-noise ratio adjusted according to test performance) to obtain the signal-to-noise ratio to recognize 50% of the digit sequences, under headphones or via loudspeakers, preferably in a soundproof booth environment (which is preferred but not necessary). Ears can be tested individually or together, with test stimuli presented via headphones or loudspeakers. When the test is being conducted in noise, different types of noise (e.g., modulated noise or speech noise) can be used and speech and noise could come from the same or different directions. For testing in noise, the noise is played about 500 ms before the digit sequence and ends about 500 ms after. This time interval is the default value and could be varied by the tester depending on the purposes of the test.

During the test, participants are instructed to verbally repeat or indicate via other means (e.g., by taping digits displayed on a computer screen) the digit sequences they heard in forward or backward order. The participant can be a child aged four and above, an adolescent, or an adult.

In one implementation, a method of testing a participant's ability to accurately detect speech in the presence of background noise includes: (a) audibly presenting a series of 2 digits, 3 digits, 4 digits and/or 5 digits, in random or fixed orders, in Mandarin (may be included as one exemplary scenario, without limitation), to a first ear of the participant in quiet or in noise at a fixed signal-to-noise ratio (SNR) and (b) then the procedures are repeated for the other ear. Tests can also be conducted with signals and noise presented to the two ears simultaneously. Scores are calculated based on the percentage of digits or digit sequences repeated correctly by the participant (i.e., the listener). Alternatively, the difference in level between speech and noise to yield 50% of the digit sequences being identified correctly is measured. Sound signals can be presented by a laptop connected to a sound card (e.g., TASCAM US 2×2 soundcard) and loudspeakers such as JBL control 25-1, or any other suitable means of presenting sound to a participant, including, but not limited to a smartphone or tablet connected to loudspeakers or headphones and calibrated to deliver the signals at appropriate levels.

In the second implementation, the level of the speech or noise is fixed, normally at 65 dBA, or at other levels custom set by the tester to meet the purpose of the test. The noise level can be fixed while the speech level is varied according to the correctness of response. Alternatively, the speech level can be fixed while the noise level is varied. A series of 2 digits, 3 digits, 4 digits and/or 5 digits, in random or fixed orders, in Mandarin, are presented to a first ear of the participant in noise at a fixed signal-to-noise ratio (SNR) and then the procedures repeated for the other ear. Tests can also be conducted with signals and noise presented to the two ears. Participants are asked to repeat (verbally) or indicate via other means, the digits heard in forward or backward order. A digit sequence is determined to be correct when all digits are repeated correctly. If the participant responds correctly, the volume of speech or noise is adjusted (for example by a software configured to adjust the speech or noise by making the speech or noise quieter by for example, 2 decibels (dB) on the next trial. If the participant responds incorrectly, the speech or noise is made louder by 2 dB on the next trial. In some forms, the test includes 24 trials in one test. The starting SNR level can be custom set based on the participants' hearing or speech recognition level, and the SRT is calculated as the average SNR used to present the 5th-24th digit sequences. In some forms the number of presentations or iterations can be determined using statistical measures to maximize accuracy while minimizing test time, and the number of trials can be up to a maximum of 24. The signal-to-noise (SNR) ratios at which about 50% of the digits/digit sequences are repeated correctly is measured. Alternatively, the difference in level between speech and noise to yield 50% of the digit sequences n being identified correctly is measured.

The disclosed method advantageously provides a cognitive screening component when a series of 4 or 5 digits are presented to the participant. The difference in SRT obtained using longer digit sequences (e.g., 5-digit) and shorter digit sequences (i.e., 2, and 3-digit) is used for cognitive screening. If this difference is greater than a certain standard value (the standard value varies depending on the version of the DINT and will be determined for each language version separately), suggesting a referral for a full cognitive assessment is recommended for the participant. The standard value or cut-of score can be determined by testing a group of participants who are speaking the language as native speakers and have no known cognitive decline, or determined by measuring reaction time. The optimal cut-off scores for discriminating individuals with cognitive decline can be determined using a method such as the Youden index (J), which identifies the cut-off value that maximizes the Youden function, representing the difference between the true positive rate and the false positive rate across all potential cut-off values. SRTs of individuals exceeding this cut-of score will be regarded as indicating possible cognitive decline. Experiment 5 below (incorporated herein) provides an exemplary test method. The cut-off score is determined for each language version of the DINT.

In some forms the test can use 1-digit sequences for the screening of hearing function, in order to further reduce test time.

Another aspect of this disclosure involves an apparatus for measuring digit recognition in relation to hearing loss. The apparatus includes a speaker measuring device and a listener measuring device. The speaker measuring device includes a first pairing connection module; a first display configured to show a sequence of characters or numbers serving as test data; and a first signal transmitting module configured to transmit the test data via the first pairing connection module. The listener measuring device includes a second pairing connection module configured to electrically communicate with the first pairing connection module; a second display configured to show an indicator signal and display a virtual keypad; a receiver configured to receive the test data from the speaker measuring device; an audio recorder configured to record environmental sound data; a testing recorder configured to record a test response inputted from the virtual keypad; and a scoring module configured to compare the test response with the test data to generate score data.

In accordance with this aspect of the disclosure, a method for measuring digit recognition in relation to hearing loss is provided. The method includes steps: pairing a speaker measuring device and a listener measuring device (including real-time, pre-recorded or real-time machine-generated digits) by using a first pairing connection module of the speaker measuring device and a second pairing connection module of the listener measuring device; showing, by a first display of the speaker measuring device, a sequence of characters or numbers serving as test data; transmitting, by a first signal transmitting module of the speaker measuring device, the test data to the listener measuring device via the first pairing connection module; showing, by a second display of the listener measuring device, an indicator signal and displaying a virtual keypad; receiving, by a receiver of the listener measuring device, the test data from the speaker measuring device; recording, by an audio recorder of the listener measuring device, environmental sound data; recording, by a testing recorder of the listener measuring device, a test response inputted from the virtual keypad; and comparing the test response with the test data to generate score data.

By the above configuration, the listener user(s) can view the scores so as to gain a better understanding of how well they can hear in a listening situation (including when using and not using hearing aids, in daily life, hearing aid fitting centers, hospitals, etc.) so that communication is less difficult. This is particularly important because many older people have great difficulties understanding speech in noise when they have hearing loss or cognitive decline. Thus, understanding the specifics of their hearing loss and what effect it has on them will reduce frustrations, enhance communication in social interactions, and prevent further deterioration in cognitive function or other negative consequences (e.g., depression) associated with hearing loss and cognitive decline when hearing devices are not used.

Recording the noise together with the digits spoken will help clinicians working with an older person understand the difficulties she or he is experiencing. Older people often find it difficult to describe the scenarios in which they have difficulties communicating. When they talk to a general practitioner, an Ear-Nose-and-Throat doctor, an audiologist, or a hearing aid dispenser, these patients are not able to describe their hearing difficulties well enough for their doctor or audiologist to understand and be able to make informed, evidenced-based recommendations to help them. Furthermore, having these recordings and scores will not only help them manage their communication difficulties with hearing health providers, but also help friends and family members understand how they can assist in the process.

For example, a hearing device user likes having a meal (e.g., dim sum) at a certain restaurant that is quite noisy. He or she has difficulty understanding some of his/her friends due to the background noise. He or she may make some adjustments to the hearing devices (e.g., volume control or change the listening program). He or she may also describe the difficulties he or she has to his/her audiologist, who would make more substantial changes to the hearing device amplification parameters, based on these comments. As it is often difficult to describe exactly what has happened in the communication exchange, the audiologist is only able to make the best guess. Repeated adjustments may not successfully address the issues. Eventually, the patient may suffer from speech and hearing problems such that he or she stops socializing with his/her friends and feels isolated.

With the proposed solution disclosed herein, the hearing device user can have his or her friends speak the digits, or the digits can be presented by a loudspeaker in the restaurant and obtain scores on his or her ability to understand digits in noise. S/he can make several adjustments to the hearing device settings and obtain scores on digit recognition with these settings. S/he can then understand better which settings would yield best speech understanding. S/he can also take these scores together with the recordings to his or her audiologist for a hearing device adjustment. The audiologist can review all the information and make adjustments to the listening programs accordingly (including hearing aid adjustments), without having to depend on the user's descriptions which may be unreliable or lack essential information. The audiologist is also able to counsel the hearing device user that his or her hearing difficulty might have stemmed from him or her not being able to handle too much noise and recommend that he or she gathers with his or her friends in restaurants that yield better speech recognition scores. These results are particularly important when the hearing device users (e.g., low health literacy or cognitive decline) do not have insights to and are unable to clearly describe his/her hearing difficulties. In addition, as the test can also evaluate cognitive function, test scores will also inform the clinician, patient, and family on the impact of certain environments on communication for a patient with cognitive decline.

Another aspect of this disclosure involves a computer-implement method (CIM) for assessing a participant's ability to detect speech. The CIM involves: (i) audibly presenting between one- and up to 24-digit sequences containing two to five digits, in random or fixed orders, to the participant, (ii) scoring how many digit sequences the participant correctly identifies in a forward or backward recall order, and (iii) displaying a result on the assessment on a graphical user interface, with information to interpret the result. Digits in a digit sequence are homogenous in difficulty and/or homogenous in psychometric functions, and are interposed with a period of silence. Means for audibly presenting the digit sequences include, but are not limited to a laptop connected to a TASCAM US 2×2 soundcard and JBL control 25-1 loudspeakers.

The test in different languages, can be developed using an automated platform with prescribed steps (see Wang and Wong, 2023) to prepare the test stimuli, with and without collection of normative data. Also described is a CIM for developing test materials for assessing a participant's ability to detect speech. The CIM involves: (a1) processing digits to be included in the test materials to introduce homogeneity in difficulty, psychometric functions, or a combination thereof, wherein the digits are processed on a processing hardware platform, and test materials for assessing the participant's ability to comprehend speech selected after step (a1). To account for the participant's ethnicity and/or geographical location, the digits are obtained from one or more individuals (b1) speaking the same native language, (b2) from the same ethnic group, and/or (b3) from the same geographical region as the participant.

Processing the digits further involves generating a deep-learning artificial intelligence-based model from another set of digits data, to provide predicted adjustment levels for each digit. A new approach leveraging the power of Deep Neural Networks (DNNs) for predicting the SRTs of individual digits was used. The architecture of the DNN will primarily be composed of several fully connected layers. The depth and width of the network will be tuned during the development process to optimize performance. The final layer of the network will contain a single neuron as the here task is to predict the SRT, a continuous value. The approach begins with the extraction of a comprehensive set of acoustic features from the digit recordings. These features include, but are not limited to, the duration of the digit, pitch information, tone information, and spectrum information. Each of these features has a potential correlation with the digit's SRT and thus can be instrumental in the prediction model. The core of this approach is the implementation of a DNN to construct an SRT prediction model. This model is trained using the acoustic features of each digit as input and their corresponding experimentally determined SRTs as the target. By processing the complex, high-dimensional input data, the DNN can capture intricate relationships between the acoustic features and SRT, which are not easily discernible with traditional methods. Once trained, this DNN model can accurately predict the SRT of a given digit based on its acoustic features. This prediction can then be used to adjust the level of each digit, harmonizing the SRTs across all digits. Preferably, during the processing, any digit requiring excessive corrections will be automatically eliminated from the test material. Lastly, the test materials are one or more digit sequences having a period of silence interposed between pairs of digits in each digit sequence.

Unless otherwise defined, all technical and scientific terms used herein have the same meaning as commonly understood by one of ordinary skill in the art to which the disclosed subject matter pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 shows the average digit recognition probabilities and the estimated psychometric function curves of the 11 digits.

FIG. 2 shows the average digit sequences recognition probabilities and the estimated psychometric function curve for the four digit sequences.

FIG. 3 shows the mean better ear and worse ear pure-tone audiometric thresholds with SDs as error bars.

FIG. 4 is a Box-and-Whisker Plots of SRTs of the four digit sequences, comparing results from young and older listeners. The black dots represent the SRTs of each participant, the “+” represents mean SRT, the box represents the quartiles, and the whiskers indicate the range of SRTs.

FIG. 5 shows receiver operating characteristic curve analysis of digit 5-2, digit 5-3, and the DST for screening of cognitive impairment.

FIG. 6 shows the cutoff point of digit 5-2 for screening cognitive decline and sentence perception in noise difficulty. The 2-digit SRT of 2 participants who failed to complete the 5-digit SRT and SIN test and failed the MoCA, were 4.7 and 7.5 dB.

FIG. 7 shows receiver operating characteristic curve analysis of 2-digit, and 3-digit for screening of sentence perception in noise difficulty.

FIG. 8 depicts a schematic diagram of an apparatus for measuring digit recognition in relation to hearing loss in accordance with various embodiments of the present invention.

FIG. 9A depicts a workflow for a hearing loss assessment program at phase I in accordance with an embodiment of the present invention. FIG. 9B depicts a workflow for a hearing loss assessment program at phase II in accordance with an embodiment of the present invention.

FIG. 10 shows how the hearing loss assessment program works for measuring speech understanding at a scenario with bare ears in accordance with one embodiment of the present invention.

FIG. 11 shows how the hearing loss assessment program works for measuring speech understanding at a scenario with a hearing device in accordance with one embodiment of the present invention.

FIG. 12 is a flow chart of an exemplary procedure involved in developing a standardized version of the iDIN test to create the test materials.

FIG. 13 is a flow chart of an exemplary procedure involved in developing a standardized version of the iDIN test to administer the test.

FIG. 14 is a flow chart of the steps for an exemplary procedure involved in creating a non-standardized version of the test materials.

### DETAILED DESCRIPTION OF THE INVENTION

Individuals with hearing loss have difficulties understanding speech. The issue is many of these individuals are also older and experience declines in cognitive function. When older adults have difficulties understanding speech, GPs, gerontologists, ENTs and audiologists have difficulties telling apart whether the difficulties stem from age-related hearing loss alone or in conjunction with cognitive decline. When benefits from hearing devices (e.g., hearing aids, cochlear implants) are poorer than expected and adjustment of hearing devices do not yield better benefit, ENTs and audiologists may wonder about the impact of cognitive function. However, ENTs and audiologists do not normally administer diagnostic cognitive function tests in clinical settings and referrals may not be taken up readily. Even if cognitive function is measured, because the tests were developed without regards for the presence of a hearing loss, test results cannot be interpreted in light of a hearing loss. Therefore, it is still difficult to know the additional impact cognitive function causes.

Thus, although digits have been used in previous research in evaluating speech understanding and digits have also been used as a cognitive function test to evaluate working memory, the disclosed methods provide a single platform integrating the evaluation of both functions, so that the effects of cognitive decline on speech understanding, in addition to the impacts of hearing loss, can be measured.

Currently, 2 to 5 single-digit sequences are used as test stimuli. SRT can be obtained based on forward repetition of 2 or 3 digits (repeating the digits as heard) for hearing screening and be used as the baseline to compare with other test conditions. Digits can also be repeated in reverse order (backward SRT). Digits duration can also be compressed (e.g., 200 ms vs 100 ms) to evaluate the ability to handle fast speech. By comparing forward 2 or 3 digit SRT and long digit sequences (e.g., 5 digits), backward SRT, and compressed SRT, variations in auditory process and cognitive function could be assessed.

### I. Definitions

The “speech recognition threshold (SRT)” refers the level at which half of speech test material could be repeated correctly.

The Digit in Noise test behaviorally measures speech understanding difficulty using different digit sequences. Comparison of results obtained using shorter digit sequencies with those obtained using longer digit sequences provides an indication of whether there is a presence of cognitive decline. That is, in the absence of a cognitive decline, the ability to repeat short and long digit sequences should not differ significantly. With cognitive decline (e.g., reduction in working memory capacity), significant reduction in performance is expected with longer digit sequences.

The term “electrically communicating/communication” as used in this patent is intended to encompass various modes of communication, including but not limited to wireless communication, wired communication, wireless pairing, and wired pairing. The scope of “electrically communicating/communication” extends to both wireless and wired methods of data exchange, wireless matching processes, and wired matching processes.

### II. Testing Methods, Apparatuses, Computer-Implemented Methods/Systems

### A. Testing Methods

The testing method uses digits, in any language. Digits are over learned, redundant speech stimuli that can be easily recognized auditorily by children and adults, regardless of education background, culture and language. In other words, it is presumed that the use of digits would be less affected by these factors, compared to traditional test materials that use words or sentences to evaluate speech understanding, thereby making digits suitable for use with individuals of all ages and background. If a mild cognitive decline is involved, the impact on recognizing short digit sequences should be minimal. Thus, the ability to recognize short digit sequences is assumed to mostly reflect an individual's auditory function, whereas cognitive decline may disrupt the ability to remember longer sequences (e.g., >3 digits) and thus SRT measured using longer sequences will be significantly poorer. This test is simple and quick to administer, as well as interpret, in clinical settings and can be extended to other settings (e.g., GP clinics, aged care facilities) to screen older adults for cognitive decline and facilitate referral.

In one implementation, a method of testing a participant's ability to accurately detect speech in the presence of background noise includes: (a) audibly presenting a series of 2 digits, 3 digits, 4 digits and/or 5 digits, in random or fixed orders, in a language the participant typically speaks/is fluent in, for example, Mandarin, Cantonese, English, French, Spanish, Japanese, Portuguese, etc., to a first ear of the participant in quiet or in noise at a fixed signal-to-noise ratio (SNR) and then the procedures can be repeated for the other ear. Tests can also be conducted with signals and noise presented to the two ears simultaneously. Scores are calculated based on the percentage of digits or digit sequences repeated correctly by the listener. Alternatively, the difference in level between speech and noise to yield 50% of the digit sequences being identified correctly is measured.

In the second implementation, the level of the speech or noise is fixed, normally at 65 dBA, or at other levels custom set by the tester to meet the purpose of the test. The noise level can be fixed while the speech level is varied according to the correctness of response. Alternatively, the speech level can be fixed while the noise level is varied. A series of 2 digits, 3 digits, 4 digits and/or 5 digits, in random or fixed orders, for example, in Mandarin, will be presented to a first ear of the participant in noise at a fixed signal-to-noise ratio (SNR) and then the procedures repeated for the other ear. Tests can also be conducted with signals and noise presented to the two ears (or tests can also be conducted with one ear with speech or noise first, then both ears are tested with speech or noise simultaneously). Participants are asked to repeat or indicate via other means the digits heard. A digit sequence is determined to be correct when all digits are repeated correctly. If the participant responds correctly, the volume of speech or noise is adjusted (for example by a custom software configured to adjust the speech or noise by making the speech or noise quieter by 2 decibels (dB) on the next trial. As an example, the Oldenburg Measurement Applications Copyright© 2022 Hörzentrum gGmbH can be used. The “Oldenburger Messprogramme” software provides audiologists and hearing care professionals with an instrument that allows them to perform adaptive measurement procedures such as sentence tests to determine the speech recognition threshold or loudness scaling quickly, conveniently and in a modular fashion (https://www.hz-ol.de/en/oma.html). If the participant responds incorrectly, the speech or noise is made louder, for example, by 2 dB on the next trial. In some forms the test uses 24 trials in one test. The starting SNR level can be custom set based on the participants' hearing or speech recognition level. The SRT is calculated as the average SNR used to present the 5th-24th digit sequences. In some forms, the number of trials are determined using statistical measures to maximize accuracy while minimizing test time, and the number of trials can vary, up to a maximum of 24. The signal-to-noise (SNR) ratios at which about 50% of the digits/digit sequences are repeated correctly are measured or the difference in level between speech and noise to yield 50% of the digit sequences being identified correctly is measured.

Sets of digits are presented to the listener in order. The set of digits is followed by a response period. The response period of each set is the default value and could be varied by the tester depending on the purposes of the test. The participant's task involves listening to the series of digits presented individually to the left and right ear, and then repeating the digits back (in forward or backward order) during the response period. The digits are presented in the presence of background noise as stated above.

The participant's responses can be made/registered in the form of written responses, oral responses, button-pushing responses (e.g., through a user interface), and by any other suitable form. The participant can receive one or more scores pertaining to the participant's performance. For example, in fixed SNR measurement, if the participant correctly identified 12 of the 24 digit sequences that were presented during the participant's performance, the participant can receive a score of 50% (12/24×100%). Other types of statistical scoring can also be used.

In some embodiments, the test is used to determine the speech reception threshold (SRT), which is the signal to noise ratio at which the participant can correctly identify 50% of the digit sequences. A speech reception threshold (SRT) to short digit sequences that is worse than the normative data, in the presence of a hearing loss (defined using traditional audiometric measures) indicates that the individual has greater difficulties with speech understanding due to the hearing loss. A speech reception threshold (SRT) to long digit sequences that is significantly worse than that for shorter sequences suggests additional difficulty with speech understanding that is attributed in this test, to cognitive decline. A speech reception threshold (SRT) to long digit sequences that is worse than that at short digit sequences, without the presence of a hearing loss, is attributed in this test to speech understanding difficulties due to cognitive decline.

### B. Apparatuses

Another aspect of the present disclosure relates to scenarios on how to assist individuals who require help with hearing, referred to as a “listener” in this disclosure. The entity transmitting a sound message to the listener is identified as a “speaker” in this disclosure. The speaker can be represented by various entities or objects. For instance, the speaker may be a human (e.g., a friend of the listener) or a machine (e.g., a loudspeaker), presenting pre-recorded digit sequences at a fixed volume or at levels adjusted based on the accuracy of the listener users' responses.

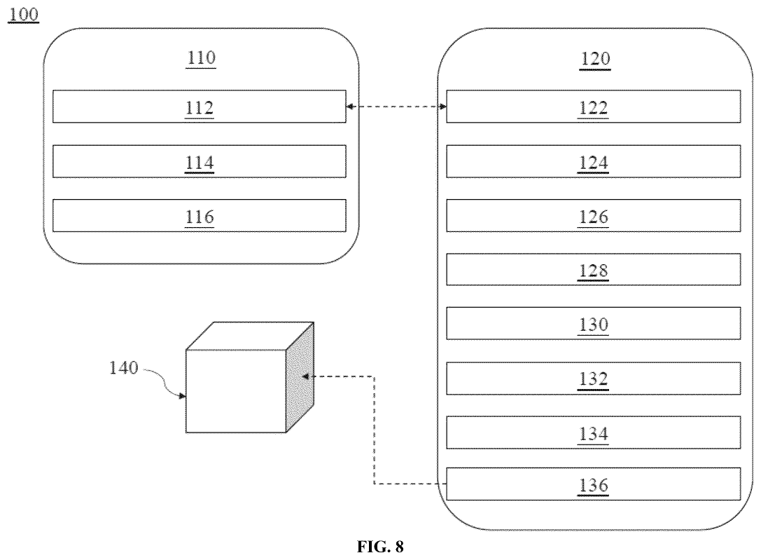

Referring to FIG. 8, apparatus 100 for measuring digit recognition in relation to hearing loss is described. The apparatus 100 includes a speaker measuring device 110 and a listener measuring device 120, through which users can interact to perform hearing tests, record results, and score the hearing tests, providing reports on hearing health. The speaker measuring device 110 and the listener measuring device 120 can be installed or implemented on handheld electronic devices, such as mobile phones, smartphones, tablets, or portable electronic devices.

The speaker measuring device 110 includes a first pairing connection module 112, a first display 114, and a first signal transmitting module 116. The listener measuring device 120 includes a second pairing connection module 122, a second display 124, a receiver 126, an audio recorder 128, a testing recorder 130, a scoring module 132, a player module 134, and a second signal transmitting module 136.

The speaker measuring device 110 and the listener measuring device 120 can be paired for electrically communicating with each other via connection between the first pairing connection module 112 and the second pairing connection module 122. In an embodiment, the connection between the speaker measuring device 110 and the listener measuring device 120 is wireless; for example, a Bluetooth channel is applied to the connection.

The first display 114 is configured to show a sequence of characters or numbers serving as test data, which visually informs the speaker user about the test data for the hearing loss assessment program.

The first signal transmitting module 116 is configured to transmit the test data to the listener measuring device 120 via the first pairing connection module 112.

The second display 124 is configured to display an indicator signal and a virtual keypad. Since the listener measuring device 120 is designed for a listener user with hearing difficulties, the indicator signal visually informs the listener user when the hearing loss assessment program is ready. The virtual keypad may contain number buttons for the listener user's input.

The receiver 126 is configured to receive the test data from the first signal transmitting module 116 of the speaker measuring device 110. In one embodiment, the receiver includes a cache to store the test data for future access.

The audio recorder 128 is configured to record environmental sound data during the hearing loss assessment program, including spoken content by the listener user embedded in surrounding background noise.

The testing recorder 130 is configured to record a test response input via the virtual keypad. Specifically, during the hearing loss assessment program, the listener user can respond to the test content by pressing the number buttons on the virtual keypad. These inputs, generated by pressing the number buttons, serve as the test responses of the listener user.

The scoring module 132 is configured to compare the test response with the test data, generating score data. As the apparatus is designed for a hearing loss assessment program, discrepancies between the test response and the original test data can be quantified and presented as score data in a hearing loss assessment report.

The play module 134 is configured to emit an indicator sound, such as “beep” sounds, for notification purposes. The sound intensity of the indicator sound is adjustable, and it can be increased when the listener user cannot clearly hear the sound. In one embodiment, when the listener user cannot clearly hear the sound, the sound intensity of the indicator sound can be increased at fixed intervals (e.g., 10 dB) until the listener user can hear it. In one embodiment, the indicator sound can be emitted simultaneously with the indicator signal.

The second signal transmitting module 136 is configured to package the score data and the environmental sound data and then transmit them via wireless communication to other devices, serving a test report 140. In an embodiment, the second signal transmitting module 136 is further configured to signal the speaker measuring device 110 as feedback according to the score data, in which the feedback can trigger the first display 114 with showing adjustment indicators. This is designed to facilitate restarting the program when program does not perform as expected.

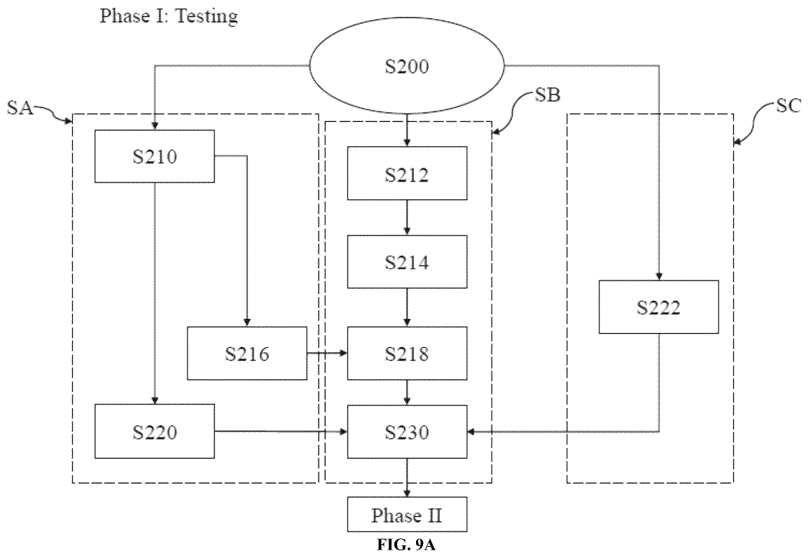

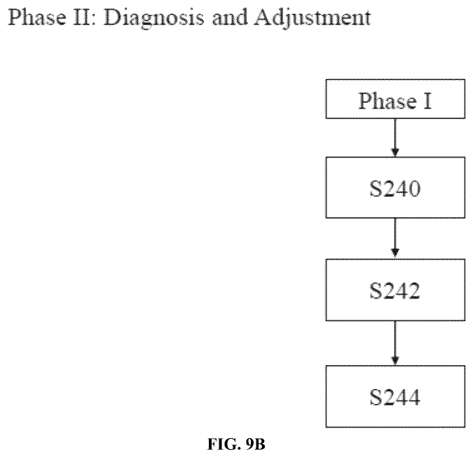

Referring to FIGS. 9A and 9B, an example workflow of a collaboration amongst components of the apparatus 100 is described.

The phase I is related to testing of the hearing loss assessment program, including steps S200, S210, S212, S214, S216, S218, S220, S222, and S230, in which the steps S210, S216, and S220 are grouped into a speaker part SA; the steps S212, S214, S218, S230 are grouped into a listener part SB; and the step S222 is grouped into an environment part SC.

The speaker user uses the speaker measuring device 110 as described above, while the listener user employs the listener measuring device 120 as previously described. In one embodiment, both the speaker measuring device 110 and the listener measuring device 120 are smartphones, and the hearing loss assessment program is operated using an application (i.e., an APP). The listener can either directly listen to the sound from the listener measuring device 120 (i.e., by bare ears), or choose to use a hearing device or earphones to receive the sound from the listener measuring device 120.

In the step S200, the speaker measuring device 110 and the listener measuring device 120 are paired, and the speaker user and the listener user can set up in a chosen environment, such as at home, in stores, or at a restaurant. In one embodiment, the hearing device or the earphones can be paired with the listener measuring device 120 as well. The speaker measuring device 110 and the listener measuring device 120 are connected via a wireless connection, such as a Bluetooth channel; for example, the APP may establish a Bluetooth channel between the speaker measuring device 110 and the listener measuring device 120.

In the step S210, the first display 114 of the speaker measuring device 110 can show a sequence of characters or numbers for the speaker user, serving as test data. In an embodiment, the test data includes a digit sequence.

In the step S212, the player module 134 of the listener measuring device 120 emits indicator sound (e.g., “beep” sounds) at increasing fixed intervals (e.g., 10 dB intervals) until indicated audible by the listener user, adjusting the sound intensity of the indicator sound so as to ensure the listener user is able to hear the indicator sound.

In the step S214, the second display 124 of the listener measuring device 120 flashes for serving as an indicator signal for the listener user and emits the indicator sound (e.g., “beep” sounds) with the sound intensity above the final audible indicator sound in the step S212 (e.g., more than at least 20 dB), to signal start of the hearing loss assessment program. In an embodiment, the indicator sound is emitted simultaneously with the flashes of the indicator signal.

In the step S216, the first display 114 of the speaker measuring device 110 shows a sequence of characters or numbers serving as test data for the speaker user to read naturally. In the step S218, the second display 124 of the listener measuring device 120 shows a virtual keypad, and then the listener user can record what he/she hears on the listener measuring device 120 via the keypad. In an embodiment, the testing recorder 130 of the listener measuring device 120 can record the input from the virtual keypad for serving as a test response for the hearing loss assessment program. As the cooperation of the steps S216 and S218, digits are read by a communication partner (i.e., the speaker user) in real-life situations (e.g., in a restaurant), while the listener user will indicate the digits heard by tapping the corresponding number displayed on a keypad on the listener measuring device 120.

In the step S220, the first signal transmitting module 116 of the speaker measuring device 110 transmits the test data to the listener measuring device 120 via the first pairing connection module 112, such that the receiver 126 of the listener measuring device 120 can receive the test data from the speaker measuring device 110. In an embodiment, digit sequence information is sent to listener measuring device 120 for scoring; that is, the listener measuring device 120 can be indicated which digits should be spoken and score the responses according to the listener user's input.

In the step S222, during the interacting between the speaker part SA and the listener part SB (i.e., during the hearing loss assessment program), the audio recorder 128 of the listener measuring device 120 can record environmental sound data, which contains the surrounding background noise and the spoken digits embedded in the surrounding background noise.

In the step S230, the scoring module 132 of the listener measuring device 120 compares the test response of the listener user with the test data to generate score data. In an embodiment, the results of the hearing loss assessment program and the sound file recorded during the hearing loss assessment program from environment are saved on the memory of the listener measuring device 120. The listener user can view the scores so to gain a better understanding of how well he or she can hear in a listening situation and compare the scores in different situations. This does not only help the listener user to gain a better understanding of how well he or she can hear but also empowers him or her with the ability to choose situations so that communication is less difficult.

In an embodiment, according to the score data, the second signal transmitting module 136 can signal the speaker measuring device 110 as feedback, triggering the first display 114 with showing adjustment indicators. This is designed to facilitate restarting the hearing loss assessment program when it does not perform as expected. For example, if the listener user with the hearing device (which is in electrical communication with the listener measuring device 120 as well) gets any of the numbers presented via the loudspeaker in a digit sequence repeated wrongly and then the scoring module 132 indicates a signal for a wrongly repeating condition, the signal level will be adjusted up by signaling the hearing device by the second signal transmitting module 136; and when the digit sequence is repeated correctly and then the scoring module 132 indicates a signal for a correct repeating condition, the signal level will be adjusted down by signaling the hearing device by the second signal transmitting module 136, such that the average level at which these digit sequences are being presented will be recorded as the threshold for digit recognition. In another application, the digit sequences will be presented at fixed levels via the loudspeaker or spoken by the friends at levels at his/her discretion and the percent correct for repeating the digits will be recorded (e.g., a correct percentage for the test response relative to the test data).

The package of the results of the hearing loss assessment program and the sound file recorded during the hearing loss assessment program from environment serves as a hearing loss assessment report. When the report is ready, the workflow enters the phase II. The phase II is related to diagnosis and adjustment according to the phase I, including steps S240, S242, and S244.

In step S240, the test results and sound file are sent to the audiologist. In an embodiment, the listener user has an account linked with his/her audiologist's account on the user interface of the listener measuring device 120 via wireless communication, such as cloud pathway for easy transferring the files. In the step S242, the audiologist interprets results and reviews sound file, presents diagnoses to the listener user. In the step S244, the audiologist gives recommendations and/or adjusts hearing devices, conducting further testing if necessary.

With the above configuration, it is possible to evaluate speech understanding in real-life scenarios anytime and anywhere. In an embodiment where the listener measuring device 120 is a smartphone, the hearing devices or earphones worn by the listener user will be paired with the listener user's smartphone to record the noise and digits heard. If hearing devices are worn, the listener user can adjust the hearing devices to optimize listening before each test is taken. The settings of the hearing devices will be recorded, so that the scores for the corresponding recordings and hearing device settings are available for analysis and comparison by the hearing device user or their audiologists/hearing device dispensers. Based on this information, appropriate adjustments to the hearing devices can be made at a clinic to optimize speech understanding in real-life situations (i.e., acoustic environments and specific talkers) that matter most to the hearing device user.

The following descriptions will further explain the situations of bare ears and wearing hearing devices, respectively.

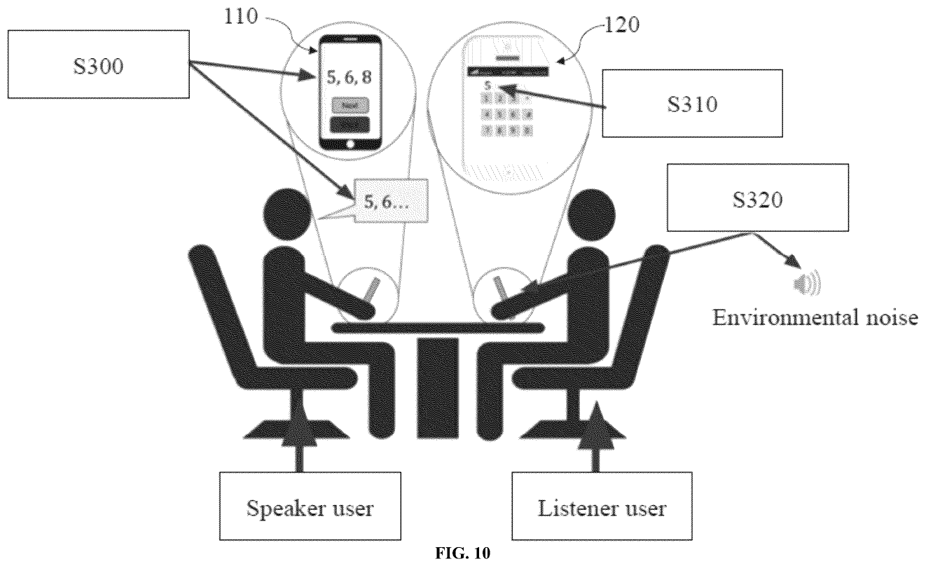

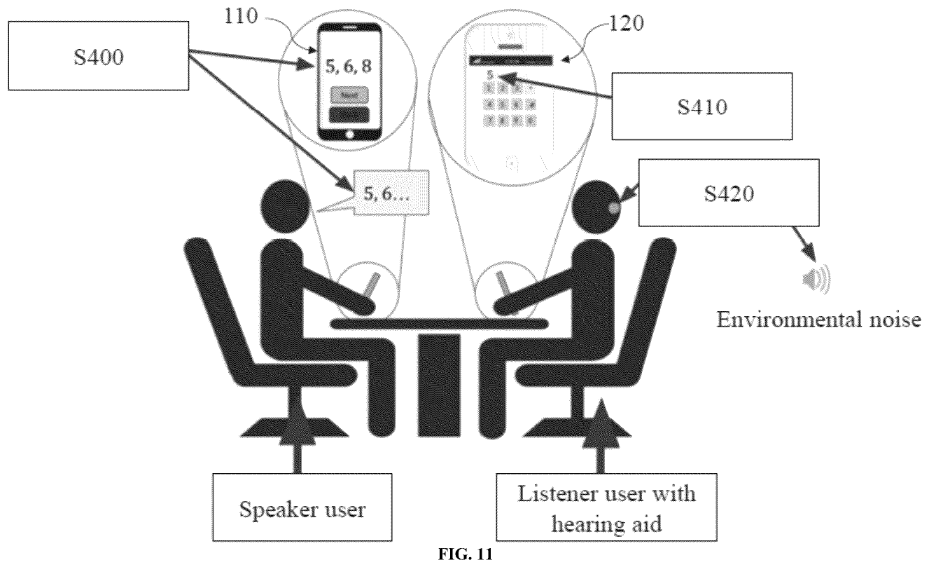

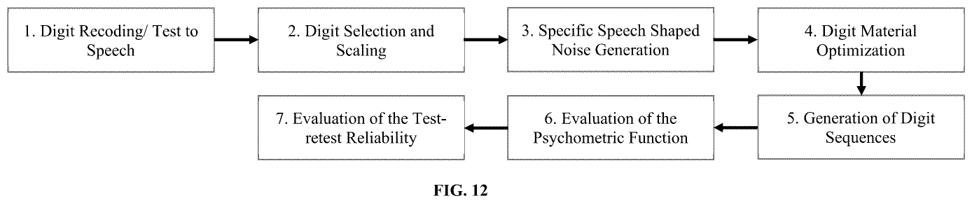

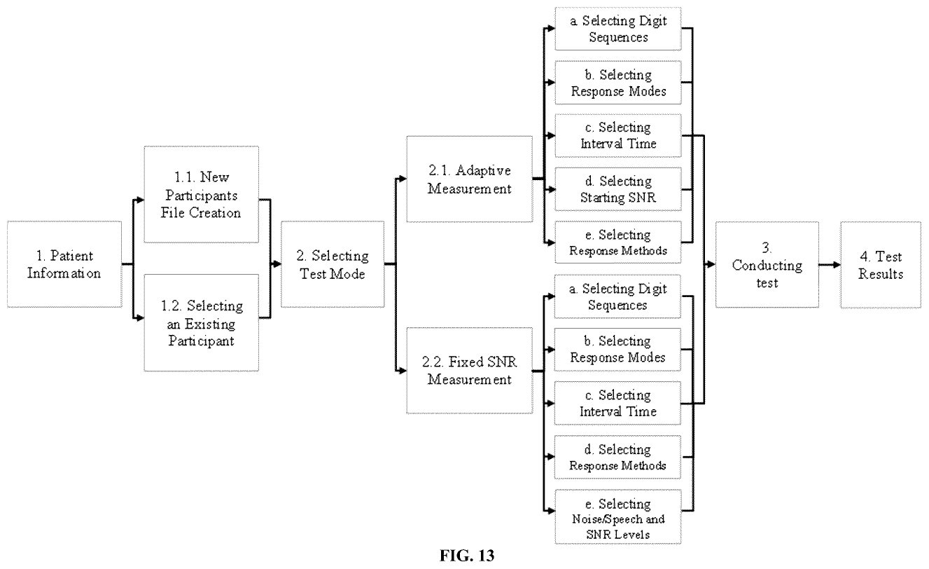

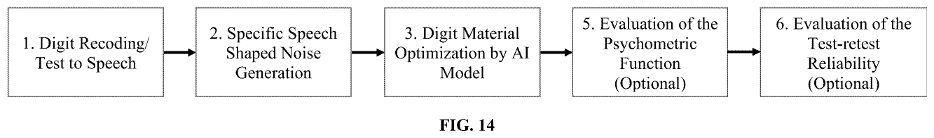

FIG. 10 shows how the hearing loss assessment program works for measuring speech understanding at a scenario with bare ears in accordance with one embodiment of the present invention. The hearing loss assessment program involves between a speaker user (loudspeaker or a human talker/friend) and a listener user and may include step S300, S310, and S320. In the step S300, the speaker user speaks the numbers displayed by the speaker measuring device 110. In the step S310, the listener user inputs digits heard from the speaker user (loudspeaker or a human talker/friend) and then the listener measuring device 120 provides the score in response to the input of the listener user. In the step S320, during the interaction between the speaker user and the listener user, the listener measuring device 120 records speech of the listener user and the environmental noise.

FIG. 11 shows how the hearing loss assessment program works for measuring speech understanding at a scenario with a hearing device in accordance with one embodiment of the disclosure. The hearing loss assessment program involves between a speaker user and a listener user equipped with a hearing device and may include step S400, S410, and S420. In the step S400, the speaker user speaks the numbers displayed by the speaker measuring device 110. In the step S410, the listener user inputs digits heard from the speaker user and then the listener measuring device 120 provides the score in response to the input of the listener user. In the step S420, during the interacting between the speaker user and the listener user, the listener measuring device 120 or the hearing device records speech of the listener user and the environmental noise.

Briefly, for people who wear hearing devices, operations in FIG. 10 will be used, except speech and environmental noise could also be recorded by the person's hearing devices, such as hearing aids, cochlear implants, (see the step S420). The hearing device user can also adjust their hearing devices (e.g., volume or other features as provided by the hearing device manufacturer) to improve listening and the hearing loss assessment program test be retaken (e.g., retaking the digit test described in this application with the improved accuracy). The listener measuring device 120 will store synchronous information that includes the test scores (see the step S410), the recordings of the test environment (see the step S420), and the corresponding hearing device settings. The hearing device user can provide the information to his/her audiologist or hearing device dispenser, so the hearing care provider can understand and adjust the hearing devices to yield the best speech intelligibility.

As described above, the devices (e.g., smartphones) are paired to enable the app/program to score responses. Tests can be conducted at any time, with any speaker, and in various environments, whether quiet or noisy. The listener has the option to listen to the digits without any hearing devices, or with hearing devices or other hearing assistance tools, to evaluate their hearing performance with these devices. This process can be repeated across multiple listening environments with different speakers to assess communication ability in diverse situations and determine the optimal hearing device settings for the best results.

The functional units and modules of the apparatuses, systems, and/or methods in accordance with the embodiments disclosed herein may be implemented using computer processors or electronic circuitries including but not limited to application specific integrated circuits (ASIC), field programmable gate arrays (FPGA), microcontrollers, and other programmable logic teaching aids configured or programmed according to the teachings of the present disclosure. Computer instructions or software codes running in the computing teaching aids, computer processors, or programmable logic teaching aids can readily be prepared by practitioners skilled in the software or electronic art based on the teachings of the present disclosure.

The embodiments may include computer storage media, transient and non-transient memory teaching aids having computer instructions or software codes stored therein, which can be used to program or configure the computing teaching aids, computer processors, or electronic circuitries to perform any of the processes of the present invention. The storage media, transient and non-transient memory teaching aids can include, but are not limited to, floppy disks, optical discs, Blu-ray Disc, DVD, CD-ROMs, and magneto-optical disks, ROMs, RAMs, flash memory teaching aids, or any type of media or teaching aids suitable for storing instructions, codes, and/or data.

### C. Computer-Implemented Methods/Systems

i. Administration of the Test

Disclosed is a computer-implement method (CIM) for assessing a participant's ability to detect speech, the CIM involving: (i) audibly presenting one or more digit sequences containing two or more digits, in random or fixed orders, to the participant, (ii) scoring how many digit sequences the participant correctly identifies, and (iii) displaying a result on the assessment on a graphical user interface, optionally with information to interpret the result, wherein, digits in a digit sequence are homogenous in difficulty and/or homogenous in psychometric functions. Preferably, pairs of digits within the one or more digit sequences are interposed with a period of silence. Preferably, step (i) involves audibly presenting one or more digit sequences containing two to five digits to the participant. In some forms, between one and 24 digit sequences, as described herein, can be audibly presented to the participant. Means for audibly presenting the digit sequences include, but are not limited to a laptop connected to a TASCAM US 2×2 soundcard and JBL control 25-1 loudspeakers.

The one or more digit sequences are presented to the participant in order. Each digit sequence is followed by response period. The response period of each digit sequence is a default value and could be varied by the tester depending on the purposes of the test. The participant's task involves listening to the digit sequence presented, optionally to the left and right ear individually, and then repeating the digits back (in order) during the response period. Optionally, the digits are presented in the presence of background noise.

In some forms, the CIM is as described above, except that the two or more digits in step (i) are obtained or processed from one or more individuals (a) speaking the same native language, (b) from the same ethnic group, and/or (c) from the same geographical region as the participant.

In some forms, the CIM is as described above, except that in step (ii), a digit sequence is scored as correct when the participant correctly repeats all digits in the digit sequence. In some forms, the CIM is as described above, except that in step (ii), the participant identifies a digit sequence presented by repeating a digit sequence aloud or inputting a response using a keypad. In some forms, the CIM is as described above, except that in step (ii) the participant can repeat the digit sequence either in a forward or backward order. In some forms, the CIM is as described above, except that in step (ii) a correct response leads to a reduction in signal-to-noise by a set decibel value (e.g., 2-dB) and an incorrect response leads to an improvement in signal-to-noise by another set decibel value (e.g., 2-dB). In some forms, the CIM is as described above, except that assessing the participant's ability to detect speech is performed via an adaptive speech recognition threshold (SRT) mode or a fixed signal-to-noise ratio (SNR) mode.

In an exemplary form, the steps for administering the test materials include:

### 1. Patient Information

Patient information will be entered into the app. These information will be used to identify the testees or calculate correlation factors for evaluating test results.

### 2. Selecting Test Mode

There will be multiple test designs, to meet the purpose of test, for the tester to select and customize. First, there are two test methods that can be selected, namely 2.1 adaptive SRT measurement and 2.2 Fixed SNR measurement. In adaptive measurement, the SNR is dynamically adjusted during the test based on the listener's performance, while in fixed SNR level measurement, a constant SNR level is maintained throughout the test.

In the adaptive SRT measurement, several test parameters can be adjusted as follows:

a. Selecting digit sequences: The test allows for the selection of digit sequences ranging from 2 to 5 digits in length.

b. Selecting response modes: Listeners can be instructed to repeat the digit sequence either in the forward or backward order. The choice of response mode is closely tied to the scoring algorithm employed in the test.

c. Selecting the length of the interval time: The duration of the silent interval between digits sequence can be customized according to specific requirements.

d. Selecting the initial SNR level: At the outset of the test, the SNR level can be tailored to suit the desired testing conditions.

e. Selecting the response method: The test provides support for two response methods, namely repeating the digit sequence aloud or inputting the response using a keypad.

In the fixed SNR level measurement test, the following parameters need to be determined:

a. Selecting digit sequences: The test allows for the selection of digit sequences ranging from 2 to 5 digits in length.

b. Selecting response modes: Listeners can be instructed to repeat the digit sequence either in the forward or backward order. The choice of response mode is closely tied to the scoring algorithm employed in the test.

c. Selecting the length of the interval time: The duration of the silent

interval between successive digits can be customized according to specific requirements.

d. Selecting the response method: The test provides support for two response modes, namely repeating the digit sequence aloud or inputting the response using a keypad.

e. Selecting the noise/speech level and SNR level: The levels of background noise, speech stimuli, and the desired SNR ratio should be determined for the test.

### 3. Conducting Test

The app will be used to run the tests, at a fixed SNR or as an adaptive procedure. During the test, participants will be instructed to repeat the digit sequences they understood. A digit sequence will be scored to be correct when all digits are repeated correctly. For the fixed SNR measurement, the test results will be presented as the percentage of digit sequence correctly repeated. In the adaptive measurement, the noise level will be fixed and could be custom set. A step size of for example, 2-dB will be used whereby a correct response will result in a 2-dB reduction in SNR and an incorrect response will solicit a 2 dB improvement in SNR. The starting SNR could be custom set, and the SRT is calculated as the average SNR of the 5th-24th digit sequences.

### 4. Test Results

Result will be displayed together with simple info on how the results should be interested.

ii. Creation of a Standardized Version of the Test Materials

Also described is a CIM for developing test materials for assessing a participant's ability to detect speech, the CIM involving: (a1) processing digits to be included in the test materials to introduce homogeneity in difficulty, psychometric functions, or a combination thereof, wherein the digits are processed on a processing hardware platform. In some forms, the CIM is as described above, except that the CIM further involves selecting the test materials for assessing the participant's ability to comprehend speech after step (a1).

In some forms, the CIM is as described above, except that the digits are obtained from one or more individuals (b1) speaking the same native language, (b2) from the same ethnic group, and/or (b3) from the same geographical region as the participant.

In some forms, the CIM is as described above, except that processing the digits involves computing adjustment levels for each digit employing an algorithm.

In some forms, the CIM is as described above, except that processing the digits further involve generating a deep learning artificial intelligence based model from another set of digits data, to provide predicted adjustment levels for each digit.

In some forms, the CIM is as described above, except that the predicted adjustment levels for each digit are based on the digit's spectral information and acoustic features, such as pitch, vowel intensity, vowel duration, and spectrum tilt.

In some forms, the CIM is as described above, except that processing each digit's sound intensity comprises adjusting the sound intensity based on the predicted adjustment value provided by the artificial intelligence model. During the processing, any digit requiring excessive corrections will be automatically eliminated from the test material. “Excessive corrections” refer to corrections that involve an adjustment level exceeding 5 decibels, 10 decibels, or 15 decibels. In preferred forms, any digit requiring an adjustment level exceeding 5 decibels will be automatically eliminated from the test material.

In some forms, the CIM is as described above, except that the test materials are one or more digit sequences having a period of silence interposed between pairs of digits in each digit sequence.

In an exemplary form, the steps for creating a standardized version of the test materials include:

### 1. Digits Recording/Text to Speech

Native speakers of a language can be recruited as talkers and digits will be spoken and recorded using set procedures. In some forms, text to speech may also be used if good recordings are available. The microphone can be placed about 20 cm directly in front of the mouth of the talker. The talkers can be instructed to speak the digits, a carrier phrase, “the digit (x)”, before the digits, up to 10 times each, into a professional quality microphone, using a natural intonation and voice, with pauses between each digit. The recordings can be saved as individual files. In some forms, the app can have the capability to record from up to 5 talkers. Alternatively, files of good quality text to speech stimuli can be adopted. After the recording, the app will automatically remove the carrier phrase and retain the recordings of the digits, by examining the rise and fall times of the digits in each recording.

### 2. Digit Selection and Scaling

The researcher/clinician/research assistants can use the app to replay the recorded stimuli and provide ratings on the naturalness, pronunciation, intonation, and speech rate of each digit stimulus. The app can select the best rated digit stimuli to be used in the actual test. Each selected digit stimulus can be scaled to the same root mean square (RMS) value, calculated as the average RMS of all stimuli. The RMS level is a measure of the magnitude of the audio signal, often used as a proxy for the audio's overall loudness. The RMS level of a signal is calculated by squaring all the sample values, calculating the mean (average) of these squared values, and then taking the square root of this mean. The RMS level for a single digit audio file can be calculated using the following formula:

$$RMS = sqrt [ ( 1 / N ) * \sum ( x\_i ) \hat{} 2 ]$$

where:
‘sqrt’ denotes the square root function
N is the total number of samples in the audio file
2 denotes the sum over all samples
x_i is the i-th sample in the audio file

### 3. Noise Generation

Randomized superposition of all digits can be used to generate a long-term average speech spectrum shaped (LTASS) noise. The noise can be played 500 ms before the digit sequence and end 500 ms after.

### 4. Digits Material Optimization

To ensure that all test stimuli have homogeneous psychometric functions, the app can evaluate the percent correct intelligibility of each digit at 11 fixed signal-to-noise ratios (SNRs) (i.e., from −2 to −22 dB in 2 dB increments). Each test list can be administered in a fixed order from −2 to −22 dB SNR, and the digits can be played back in random order at each SNR. The noise can be fixed at 65 dBA and started 500 ms before the first digit and ended 500 ms after the last digit. Participants can listen monaurally via a pair of audiometric headphones or earphones connected to the app and repeat the digits that they understood. A logistic function:

$$I ( SNR ) = y + ( 1 - y ) \frac{1}{1 + {e}^{4 s ( S R T - S N R )}}$$

can be used to determine the psychometric function of each digit using a maximum likelihood procedure, where SI=digit intelligibility, y=guess level, and s=slope at the SRT. The SRT and slope of each digit can be obtained, and the difference between the SRT of each digit and the average SRT of all digits can be used to adjust the level of each digit (Smits, C., Theo Goverts, S., & Festen, J. M. (2013). The digits-in-noise test: Assessing auditory speech recognition abilities in noise. J. Acoust. Soc. Am., 133(3), 1693-1706). After the adjustment, the clinician/researcher will listen to all the adjusted digits in quiet and noise, and judge whether any adjusted digit is unnaturally loud or soft. Digits that would require an exceptionally large adjustment (i.e., more than 4 dB) or sounded too soft or loud can be excluded.

### 5. Generation of Digit Sequences

The digit sequences (1-, 2- and other digit sequences) can be synthesized by combining single digit recordings. A period of silence will be interposed between each digit within a sequence. The duration of the interval can be tailored as per requirements. For each digit sequence condition (2- to 5-digit), a unique sequence test list comprising 24 digit sequences can be generated each time whereby the digits will be evenly distributed in each position of each digit sequence without repeats in a digit sequence.

### 6. Evaluation of the Psychometric Function of Each Digit Sequence

The results of at least four fixed SNR measurements can be used to fit the psychometric function curve of each digit sequence, in order to measure the slope of each iDIN test condition. At least 12 young adults with normal hearing need to participate in this step.

### 7. Evaluation of the Test-Retest Reliability of Each Digit Sequence

The results of at least two adaptive SRT measurements can be used to evaluate the test-retest reliability. The test-retest reliability is defined as the error of measurement, denoted by the root mean square of the within-participant standard deviation of the difference between test and retest, divided by √2 (Smits, C., & Houtgast, T. (2007). Recognition of digits in different types of noise by normal-hearing and hearing-impaired listeners. Int. J. Audiol., 46(3), 134-144).

iii. Alternative Non-Standardized Version of the Test Materials

In some forms, if the clinician is not able to follow the steps above to create a standardized version of the test, the clinician can use the following procedures to create a non-standardized version. Other than recording the test stimuli, which will take about 10 minutes to complete, all other steps are automated via the smart platform. If text-to-speech recordings are available, they can be uploaded for automated processing, saving the time for recording test materials.

In an exemplary form, the steps for creating a non-standardized version of the test materials include:

### 1. Digit Recording/Text-to-Speech

Clinicians/tester can utilize the recording function in the application to perform digit recording. The speaker's mouth should be positioned approximately 20 cm away from the microphone of the mobile device or laptop. The speaker will follow software prompts to individually record digits from 0 to 10. The software will monitor sound intensity, providing feedback if the sound level is too loud or too quiet. Text-to-speech can be used if quality recordings are available. Each digit should be recorded at least three times, allowing the tester to choose the best recording based on factors such as naturalness, pronunciation, intonation, and speech rate. The software will automatically trim the chosen digit recording, retaining only the essential digital spectrum information. All chosen digit stimuli will be adjusted to the same root mean square (RMS) value, calculated as the average RMS of all stimuli.

### 2. Noise Generation

A randomized superposition of all recorded digits will be used to create a long-term average speech spectrum (LTASS) shaped noise. This noise will be introduced 500 ms before and end 500 ms after the digit sequence.

### 3. Digit Material Optimization

To establish homogeneity in all test stimuli, the app will compute adjustment levels for each digit. In deviation from the standard optimization step, a deep learning-based AI model will be generated from previous data, providing predicted adjustment levels for each digit based on their spectral information and acoustic features, such as pitch, vowel intensity, vowel duration, and spectrum tilt. The software will automatically adjust the sound intensity of each digit based on the correction value suggested by the AI model. Any digit requiring excessive corrections will be automatically eliminated from the test material.

### 4. Generation of Digit Sequences

Digit sequences (including 1-, 2-, and other digit sequences) will be constructed by combining individual digit recordings. A silent interval will be interspersed between each digit within a sequence, with the duration adjustable to specific needs. For each digit sequence condition (2- to 5-digit), a unique sequence test list containing 24 digit sequences will be produced, ensuring that digits are evenly distributed within each sequence position and no repetitions occur within a single sequence.

### 5. Evaluation of the Psychometric Function of Each Digit Sequence (Optional)

The results of at least four fixed SNR measurements will be used to fit the psychometric function curve of each digit sequence, in order to measure the slope of each iDIN test condition. At least 12 young adults with normal hearing need to participate in this step. This step is optional and not obligatory for the development of non-standardized versions.

### 6. Evaluation of the Test-Retest Reliability of Each Digit Sequence (Optional)

The results of at least two adaptive SRT measurements will be used to evaluate the test-retest reliability. The test-retest reliability is defined as the error of measurement, denoted by the root mean square of the within-participant standard deviation of the difference between test and retest, divided by √2 (Smits, C., & Houtgast, T. (2007). Recognition of digits in different types of noise by normal-hearing and hearing-impaired listeners. Int. J. Audiol., 46(3), 134-144). This step is optional and not obligatory for the development of non-standardized versions.

Each of the functional units and modules in accordance with various embodiments also may be implemented in distributed computing environments and/or Cloud computing environments, wherein the whole or portions of machine instructions are executed in distributed fashion by one or more processing teaching aids interconnected by a communication network, such as an intranet, Wide Area Network (WAN), Local Area Network (LAN), the Internet, and other forms of data transmission medium.

Those skilled in the art will recognize, or be able to ascertain using no more than routine experimentation, many equivalents to the specific embodiments of the invention described herein. Such equivalents are intended to be encompassed by the following claims.

### EXAMPLES

The aim of the study was to establish a speech test that is easy to administer and interpret, and whose performance is not affected by literacy levels and age, and is able to simultaneously evaluate and separate the effects of hearing and cognitive functions. The relation between the number of digits and cognitive function (e.g., working memory) (and, therefore, the results of the DINT) was examined. Although several language versions of the DTT are available via the World Health Organization (WHO) for hearing screening, a search of the literature did not yield information about its development or psychometric properties (i.e., the validity and reliability of the measurement). Also, the test evaluates speech reception threshold (SRT) to 3-digit sequences only, as a tool to screen hearing and there is no predicament or indication that the WHO version nor any digit in noise test of any version, in any language, will be used to screen or evaluate cognitive function. Research studies were and will be conducted to develop different language versions (e.g., of the DINT), examine and establish the psychometric properties and provide the empirical evidence concerning the implication of the number of digits for use in a DINT and the potential in screening or evaluating cognitive function using a combined test.

In Wang and Wong (2023), four experiments were designed: Experiment 1 involved the development of materials for the Mandarin DINT; Experiment 2 evaluated the test-retest reliability and psychometric function of the Mandarin DINT among young normal hearing adults; and Experiment 3 expanded on the findings from Experiment 2 by examining the test-retest reliability and criterion validity of the Mandarin DINT among older adults with hearing loss and evaluating the effects of digit sequence on SRT. Experiment 4 examined how well the test could be used to screen cognitive function. Ethical approval for all four experiments was granted by the appropriate Institutional Review Board (IRB).

### Experiment 1: Development of the Mandarin Digit-In-Noise Test

The Mandarin DINT materials were developed mainly based on the recommendations of the International Collegium of Rehabilitative Audiology (ICRA) for developing speech tests in general (Akeroyd et al., 2015) and the development process of the Dutch DINT using 3-digit sequences (Smits et al., 2013) for assessing speech understanding as an auditory function. Digits 0-10 were selected and recorded, and levels were adjusted to achieve homogeneity across digits. The digits were combined into four digit (i.e., 2-digit, 3-digit, 4-digit and 5-digit) sequences.

### Materials and Method

### Selection, Recording and Processing of Digits

It is important to ensure the homogeneity of test items so that they are of equal difficulty. Because Mandarin digits from 0 to 10 are monosyllabic, they can all be used without the worry that the number of syllables could make some digits more readily identifiable (Akeroyd et al., 2015). Thus, a total of 11 digits were used. The recording was conducted in a sound-treated booth at the University of Hong Kong, using a Shure SM7B microphone fixed at 25 cm away from the speakers' mouth (Holube et al., 2010), connected to a TASCAM US-2×2 soundcard and a ThinkPad X1 carbon laptop. The digits were recorded using Adobe Audition pro software at a 44100 Hz sampling rate and 32-bits resolution.

Female speakers were selected, as an “acoustic compromise” between children and male speakers. Three native standard Mandarin-speaking voice actors, who spoke clearly and without misarticulation, at an appropriate rate and with stable vocalization effort, were recruited. Digits in most DTTs are usually recorded in a digit triplet format to retain natural coarticulation and prosody. Each digit is recorded at three positions in the triplet and then cut and resynthesized into final triplets. However, as no significant difference in SRT has been found with or without natural coarticulation and prosody (Lyzenga & Smits, 2011), test materials in the current study were recorded as single digits.

Before the formal recording, the voice actors familiarized themselves with the materials. During the recording, the voice actors were asked to use a natural intonation and voice as in everyday communication and maintain their vocalization effort as stable as possible. The voice actors read a carrier phrase, “the digit”, before the digits to ensure that the materials were read naturally. Although the voice actors were asked not to intentionally pause between the carrier phrase and digit, there was no spectral overlap between them. Any recording with unnatural intonation, inaccurate pronunciation, inappropriate speaking rate or unstable vocalization effort was rejected during the recording process. Finally, the three best versions of the 11 digits from at least 6 versions of each voice actor were selected.

Next, five native Mandarin-speaking audiologists ranked the recordings of each voice actor based on the overall quality of naturalness, pronunciation, intonation, and speech rate to determine the best voice actor (Hu et al., 2018; Wong et al., 2007). The best quality recoding was credited with 3 points and the worst 1 point, and scores from the five audiologists were aggregated to select the highest rated digits. Then, the same rating process was conducted to select the best overall quality digits from three recordings of each digit spoken by the selected voice actor. The recordings of the digits were extracted and saved using the Adobe Audition pro software. The complete spectrum of digits was preserved to ensure that the cutting would not distort the signals. A randomized superposition of all digits was used to generate a long-term average speech spectrum shaped (LTASS) noise.

### Optimization of Digits

Optimization of digits was conducted to achieve homogeneity in the intelligibility of each digit as closely as possible, resulting in an increase in the slope of the psychometric function for the test materials (Akeroyd et al., 2015).

Twenty normal hearing native Mandarin-speaking young adults aged between 21 to 29 years (M=23.9, SD=2.5) participated. Bilateral pure-tone audiometric thresholds were not worse than 20 dB hearing loss at the octave frequencies of 250 to 8000 Hz. Participants reported no tinnitus, otalgia, or other otologic disorders. Otoscopy revealed clean and normal ear canals. Demographic information concerning sex, age, education level was collected.

All tests were conducted in a sound-proof booth at the University of Hong Kong. A custom Matlab program was used to control the signals and administer and score the test. Four test lists containing the 11 digits at 11 fixed signal-to-noise ratios (SNRs) (i.e., from −2 to −22 dB in 2 dB increments) were created. Each test list was administered in a fixed order from −2 to −22 dB SNR, and the digits were playedback in random order at each SNR. The noise was fixed at 65 dBA and playedback 500 ms before the digits and ended 500 ms after the digits. Participants listened monaurally via a pair of Sennheiser HDA 300 headphones connected to a TASCAM US-2×2 soundcard. Testing started in the right ear and alternated twice between the two ears. Participants were instructed to repeat all digits they heard. A break of 2 minutes was provided between test lists.

### Results

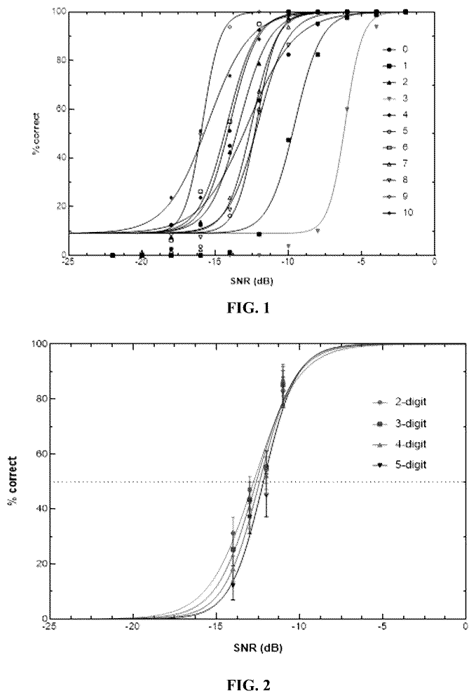

A logistic model function (Equation [1]) was used to determine the psychometric function of each digit using a maximum likelihood procedure where SI=digit intelligibility, y=guess level (in this case, y=1/11), and s=slope at the SRT. FIG. 1 shows the average recognition probabilities of the 11 digits at different SNRs.

$$SI ( SNR ) = y + ( 1 - y ) \frac{1}{1 + {e}^{4 s ( SRT - S N R )}} \qquad ( 1 )$$

After the SRT and slope of each digit was obtained, the difference between the SRT of the target digit and the average SRT of all digits was used to adjust the level of each digit (Smits et al., 2013). The adjustment levels of all digits were within +/−4 dB of the grand mean SRT, except for the digit “3”, which required a greater adjustment of 6.42 dB to achieve the same intelligibility (see the red curve in FIG. 1). After the adjustment, five native Mandarin speaking audiologists were asked to listen to all the adjusted digits in quiet and noise, and judge whether any adjusted digit was unnaturally loud or soft. All audiologists reported that the adjusted digit 3 was unusually loud compared to other digits. Therefore, the digit “3” was excluded from the final set of materials.

### Generation of Digit Sequences

The digit sequence generation followed the procedures described in the development of the Dutch DINT (Smits et al., 2013). The four digit sequences were synthesized by combining single digit recordings. A 200 ms silent interval was inserted between each digit within a sequence. A 120-digit sequence corpus was generated whereby the digits were evenly distributed in each position of each digit sequence without repeated digits in a digit sequence. Since there are two digit positions, only 90 (10×9) 2-digit sequences were generated.

A custom Matlab program was used to run the Mandarin DINT. The test can be conducted at a fixed SNR or as an adaptive procedure. According to the list generation method from Smits et al. (2013), in the adaptive DINT test trial, a list of 24 digit sequences was created using a random selection of digit sequences from a corresponding digit sequence corpus (Smits et al., 2013). In the fixed SNR measurement, a list of 20 digit sequences was created in each test trial. In the current study, the results of the fixed SNR measurement were used to fit the psychometric function curve of each digit sequence; thus, the 20 digit sequence list generation method with shorter testing time was used. The total number of digits in the 2-digit sequence test is small (i.e., 40 in the fixed SNR procedure; 48 in the adaptive procedure). The program was set such that within each list, each digit occurred at least once in different positions. The noise was played 500 ms before the digit sequence and ended 500 ms after. During the test, participants were instructed to repeat the digit sequences they heard. A digit sequence was determined to be correct when all digits were repeated correctly. For the fixed SNR measurement, the test results were presented as the percentage of digit sequence correctly repeated. In the adaptive measurement, the noise level was fixed and could be custom set. A 2-dB step size was used whereby a correct response would result in a 2-dB reduction in SNR and vice versa for an incorrect response. The starting SNR level can be custom set, and the SRT is calculated as the average SNR used to present the 5th-24th digit sequences.

### Experiment 2: Psychometric Function and Test-Retest Reliability of Each Digit Sequence Among Normal-Hearing Listeners

Psychometric properties of the four digit sequences (i.e., test-retest reliability) were evaluated among normal hearing listeners. The slope of a psychometric function indicates the maximum rate of recognition change at a particular SNR. As a steeper slope indicates better test efficiency and accuracy (Versfeld et al., 2000), the slopes were examined to select the digit sequence to be used.

### Materials and Method

### Participants and Equipment

Participants were 54 young normal hearing native Mandarin-speaking adults aged between 21 and 29 (M=24.2, SD=2.0). Bilateral pure-tone audiometric thresholds were not worse than 20 dB hearing loss at octave frequencies of 250 to 8000 Hz. Participants were undergraduate students of the Ningbo College of Health Sciences in mainland China. All tests were conducted in sound-treated booths. A laptop connected to a TASCAM US 2×2 soundcard and Sennheiser HAD 300 headphones were used.

### Procedure

Each digit sequence was played back at four fixed SNR levels (−11, −12, −13, and −14 dB SNR), which covered the 20% to 80% intelligibility range, determined via a pilot trial. The adaptive SRT test was conducted twice with each digit sequence, within the same test session, to investigate the test-retest reliability. One 3-digit DINT was provided as practice before the formal adaptive DINT. All tests were conducted binaurally under headphones in random order. Twenty participants completed the fixed SNR, and their data were used to fit the psychometric functions. The remaining 34 participants completed the adaptive DINTs, and their data were used to form normative data and calculate the measurement error. A break was offered any time participants show signs of fatigue and on request.

### Results

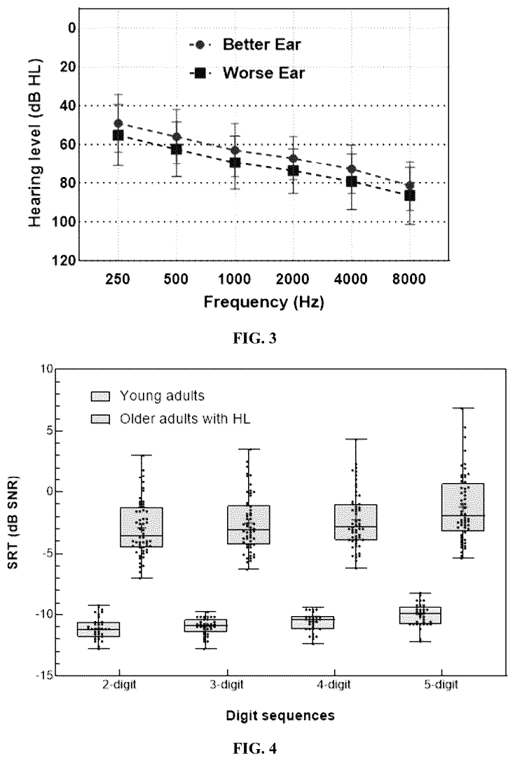

The psychometric function of each digit sequence was fitted using the logistic function Equation (1), where y=0 in this case (FIG. 2). As the number of digits increased from 2 to 5, the fitted SRT at fixed SNR became poorer, and the slope increased (Table 1). Mean SRTs of the adaptive DINT decreased as the digit sequence increased in length. Shapiro-Wilk Normality Tests showed that the data from the adaptive DINT were normally distributed. The assumption of sphericity was met, according to a Mauchlyχs test of sphericity, ×2(5)=9.08, p=0.106. An ANOVA indicated a significant digit difference effect, F(3, 99)=21.24, p<0.001, partial η2=0.39. Post hoc analysis with Bonferroni adjustment revealed that each pairwise difference was significant (ps≤0.012) except between 2-digit and 3-digit sequences (p=1.00). SRTs of the adaptive DINT were close to the fitted SRTs.

TABLE 1
Findings from testing at fixed SNRs and adaptive testing in normal hearing young adults

| | Fixed SNR Testing | | Adaptive Testing | | | |
|---|---|---|---|---|---|---|
| | | Fitted | | | Mean (of the | Measurement |
| Digit | Slope | SRT | First SRT | Second SRT | two SRTs) | Error (dB |
| Sequence | (%/dB) | (dB SNR) | (dB SNR) | (dB SNR) | (dB SNR) | SNR) |
| 2-digit | 16.58 | −11.17 | −11.03 (1.07) | −11.19 (0.85) | −11.11 (0.90) | 0.32 |
| 3-digit | 18.79 | −11.05 | −10.91 (0.82) | −11.08 (0.76) | −10.99 (0.70) | 0.28 |
| 4-digit | 20.42 | −10.89 | −10.46 (0.90) | −10.72 (0.82) | −10.56 (0.82) | 0.26 |
| 5-digit | 21.09 | −10.66 | −9.92 (0.95) | −10.13 (1.05) | −10.02 (0.95) | 0.29 |

τ=measurement error, t=test, rt=retest, k=1, . . . , n, Δk=tk−rtk;

### Values in Parenthesis Show Standard Deviations.

The test-retest reliability is defined as the error of measurement, denoted by the root mean square of the within-participant standard deviation of the difference between test and retest, divided by √2 (Smits & Houtgast, 2007) (Equation (2)). The measurement error of the four digit sequences ranged from 0.26 to 0.32.

$$τ = \frac{1}{\sqrt{2}} \sqrt{\frac{\sum {( {Δ}_{k} - {\overline{Δ}}_{k} )}^{2}}{n}} \qquad ( 2 )$$

### Experiment 3: Validation of the Dint Among Older Adult Hearing Aid Users

The relationship between digit recognition in noise and cognitive function was explored, and test-retest reliability and criterion validity were evaluated among older adult hearing aid users. The criterion validity of each digit sequence was determined by correlating the results with the SRTs obtained in the MHINT and CMNmatrix sentence test.

### Materials and Method

### Participants and Equipment

Fifty-six older native Mandarin-speaking adults aged between 60 to 87 (M=70.7, SD=6.6) were recruited through convenience sampling. The participants were bilateral hearing aid users from the Hearing Center in Beijing. All the participants had at least two years of bilateral hearing aid use experience. Participants exhibited moderate to severe sensorineural hearing loss and reported no ear diseases. FIG. 3 shows the mean audiometric thresholds of the better ear and the worse ear. The equipment was identical to that used in Experiment 2 except that the headphones were replaced by JBL Control 25-1 loudspeakers. All hearing related testing was conducted with HAs set to usual use settings, in a sound-treated booth.

### Test Materials

SRTs were measured with the DINT, the MHINT and the CMNmatrix sentence test using an adaptive procedure. The MHINT was the first standardized Mandarin sentence perception test to be established using the same paradigms as other language versions of the HINT (Wong et al., 2007). Twelve 20-sentence lists consisting of 10 characters per sentence were used. The CMNmatrix sentence test was established following the ICRA guidelines (Akeroyd et al., 2015; Hu et al., 2018). The CMNmatrix sentence test consists of semantically unpredictable and syntactically fixed sentences from a base matrix of 50 words including 10 names, verbs, numerals, adjectives, and nouns. Compared to sentences in the MHINT, which represent a conversational style of daily communication, less contextual cues are available from the CMNmatrix sentences (Hu et al., 2018; Jansen et al., 2012; Wong et al., 2007). In contrast to the HINT, which is an open set test, the CMNmatrix could be administrated as a closed or open set test.

Working memory capacity was assessed using two tests: the Mandarin version Reading Span Test (RST) and the Digit Span Test. The RST measures verbal Working memory capacity and is the most frequently used test in cognitive hearing science research (Rönnberg et al., 2019). A robust relationship between reading span and sentence in noise recognition has been established (Souza & Arehart, 2015). As the DINT to be developed in the current study employs digit sequence recognition in noise, performance on the DINT was expected to correlate with findings from the Digit Span Test, which also uses digit sequences.

In the Reading Span Test, participants would read and judge the plausibility of a sequence of short sentences in the subject-verb-object form, presented on a monitor at a rate of one word per 800 ms, with 100 ms blank screen intervals. After each sentence sequence, participants were asked to recall the first or last words of each sentence promptly. Two lists with 2-, 3-, 4-, and 5-sentence sequences were presented in random order. Test scores were determined by the number of correctly recalled words, up to a maximum of 28 points. A practice list with 2- and 3-sentence sequences was conducted before the formal test.

The Digit Span Test from the Chinese version of the Wechsler Adult Intelligence Scale (Gong, 1992) was also used to measure auditory working memory capacity. Participants were asked to listen to a series of digits and repeat them in either a forward or a reverse order at a speed of one word per 1000 ms. Like other language versions of the Digit Span Test, there were no standardized recordings of the test material. Thus, the Digit Span Test material used in the current study was generated using the single digit recordings generated in the development of the Mandarin DINT (Experiment 1). The test started with three digits in forward order and two digits in reverse order, respectively, and stopped when the participant failed to repeat the same digit length twice. The test was scored as the sum of the longest digits in the forward order and the reverse order that the participant could correctly repeat. Prior to the test, participants were familiarized with the pronunciation of all single digits used in the test by listening to a practice audio containing the digits 0-9, presented at a level that was most comfortable and yielded the best clarity.

### Procedure

The DINT, the MHINT, and the CMNmatrix sentence test were administered in sequence. Participants wore their own hearing aids set to usual use settings. Before the formal speech tests, one adaptive 3-digit DINT, one adaptive MHINT, and one open-set Matrix sentence test at 0 dB SNR were administered as practice. The four DINT digit sequences were presented in random order, with each digit sequence administered twice to participants to measure test-retest reliability. Speech and noise were presented from the front loudspeaker situated 1 meter away from the center of the head of the participants, with noise fixed at 65 dBA and speech varying in level in an adaptive procedure. Then the Digit Span Test, and RST were conducted for each participant in sequence. A break was provided whenever participants showed indications of tiredness or on request. The entire test was completed in about 90 minutes.

### Results

### Demographics

Table 2 shows the demographic characteristics, working memory test results, and SRT results for young and older adults.

TABLE 2
Demographic information, working memory test scores, and speech recognition thresholds for young and
older adults. Values in parenthesis show standard deviations. Score ranges are provided in brackets.

| | Young Adults, n = 34 | | Older Adults, n = 56 | |
|---|---|---|---|---|
| Demographic Information | | | | |
| Mean age (years) | 19.81(0.98) | [18-22] | 70.68 (−6.56) | [60-87] |
| Sex (% male/% female) | 41%/59% | | 73%/27% | |
| Education level (years) | 13.20 (−0.41) | [13-14] | 9.09 (3.53) | [0-19] |
| Better ear pure tone average | 7.27 (3.22) | [−1.25-11.25] | 64.47 (10.82) | [46.25-93.75] |
| at 0.5, 1, 2, 4 kHz (dB HL) | | | | |
| Worse ear pure tone average | 10.06 (3.26) | [1.25-12.25] | 71.16 (11.25) | [51.25-96.25] |
| at 0.5, 1, 2, 4 kHz (dB HL) | | | | |
| Hearing aid use experience (years) | NA | | 7.70 (6.30) | [2-30] |
| WM Tests | | | | |
| The Mandarin RST (score) | NA | | 11.41 (3.57) | [3-19] |
| The Digit Span Test (score) | NA | | 10.59 (1.72) | [8-14] |
| SRT | | | | |
| 2-digit DINT (dB SNR) | −11.11 (0.90) | [−12.8-−9.4] | −2.94 (2.26) | [−6.5-3] |
| 3-digit DINT (dB SNR) | −10.99 (0.70) | [−12.8-−10.2] | −2.55 (2.32) | [−6.3-3.5] |
| 4-digit DINT (dB SNR) | −10.59 (0.82) | [−12.4-−9.4] | −2.28 (2.30) | [−5.6-4.3] |
| 5-digit DINT (dB SNR) | −10.02 (0.95) | [−12-8.4] | −1.25 (2.73) | [−5.4-6.8] |
| The MHINT (dB SNR) | −6.12 (0.76) | [−7.4-−4.8] | 3.53 (2.66) | [−1.4-9.6] |
| The CMNmatrix sentence test (dB SNR) | −9.96 (0.72) | [−11.2-−7.9] | 1.60 (3.24) | [−3.8-9.4] |

CMNmatrix, Mandarin Chinese Matrix; DINT, Digit in Noise Test; Digit Span Test, Digit Span Test; MHINT, Mandarin Hearing in Noise Test; RST, Reading Span Test; SRT, speech recognition threshold; WM, working memory.

FIG. 4 shows that the mean SRTs become poorer when the digit sequence increased from 2 to 5 in older adults. A Shapiro-Wilk Normality test showed that data were not normally distributed. A Friedman test showed statistically significant difference between the digit sequences, χ2(3)=68.41, p<0.001. Post-hoc Wilcoxon tests using a Bonferroni-adjusted alpha level of 0.013 (0.05/4) revealed significant pairwise comparisons (ps<0.001). Spearman's rank-order correlation coefficients between 2- to 4-digits SRTs were 0.97 to 0.98, p<0.001 and between SRT of 5-digit and the other digit sequences were 0.88 to 0.91, p<0.001. Table 2 shows that, as the number of digits increased from two to five, SRT increased from −11.11 to −10.02 dB SNR in young adults with normal hearing and from −2.94 to −1.25 dB SNR in older adults with hearing loss. While the increase in SRT span between different digit sequences of younger listeners within 0.54 dB, the increase in SRT among older adults was greater, particularly between 4- and 5-digit sequences, at 1.69 dB. Greater variability in results was noted with older adults (SD=2.22 to 2.73 vs 0.70 to 0.90 in younger adults, across the digit sequences).

### Criterion Validity and Test-Retest Reliability

Criterion validity was examined by correlating the SRTs obtained from the DINT with those from the two sentence tests. Shapiro-Wilk Normality Tests revealed non-normal distribution of all data. Spearman's rank-order correlation coefficients ranged from 0.88 to 0.89 (p<0.001) across all digit sequences and across all participants.

Test-retest reliability was determined by examining the measurement error calculated using Equation (2). The measurement errors of the four digit sequences in older adults were 0.69, 0.71, 0.64, 0.74 dB respectively. When the young adult data in experiment 2 was included, the overall measurement errors of the four digit sequences were 0.65, 0.65, 0.61, 0.70 dB respectively.

### The Relationship Between Cognitive Function and SRT

Spearman's rank-order correlations showed DINT SRTs significantly correlated with better ear four-frequency (0.5, 1, 2, 4 kHz) pure tone average (rs=−0.29 to −0.32, ps≤0.029) and age (rs=−0.29 to −0.33, ps≤0.029) in older adults but not with the number of years of education (rs=−0.13 to −01, ps≥0.341).

Spearman's rank-order correlations were used to determine the correlation between the results of the four DINT digit sequences and the results of the Digit Span Test and the RST. With a p-value set at 0.01 due to multiple comparisons, among the four digit sequences, only the 5-digit DINT correlated with results of the Digit Span Test (rs=−0.37, p=0.005) and the RST (rs=−0.42, p=0.001). Pearson's partial correlations controlling for better ear four-frequency pure tone average and age were similar to those without controlling for these factors (i.e., r changed to −0.40 for the Digit Span Test, p=0.002 and to −0.40 for the RST, p=0.003).

### Experiment 4: A Preliminary Exploration of the Digit in Noise Test for Screening Cognitive Function in Older Hearing Aid Users

The current study will examine the impact of varying the number of digits in SRT measurement in evaluating speech perception in young NH listeners and older adults, and the sensitivity and specificity in screening the cognitive function of older adults.

### Materials and Method

### Participants and Equipment

Participants were recruited from the Shengkang Hearing Center in Beijing, China. A total of 226 hearing aid users who met the following criteria were invited via telephone calls to participate in the study: 1) have sensorineural hearing loss in both ears, 2) are native Mandarin speakers residing in Beijing; and 3) aged 60 years and above. To obtain data from participants with a range of cognitive abilities, a proportion of participants with poor cognitive performance were purposefully recruited. Eventually, 81 native Mandarin-speaking older adult hearing aid users (62 males and 19 females) aged between 60 to 95 years (M=72.51, SD=7.57, Mdn=72) agreed to participate in this study. Their mean number of years of formal education was 8.3 years (SD=3.14, Mdn=9), and the mean duration of HA use was 7.32 years (SD=5.84, Mdn=6).

### Test Materials

### Speech Perception Tests

The Mandarin DINT, the Mandarin Hearing in Noise Test (MHINT), and the Mandarin Chinese Matrix (CMNmatrix) Sentence Test were employed to evaluate the SRT in noise. SRT is defined as the SNR level at which 50% of sentences or digit sequences are correctly recognized in noise, with a one-up and one-down adaptive procedure. A long-term average speech-spectrum shaped noise for each test material was used. The sentence in noise tests were primarily employed to identify and categorize older HA users who exhibited notably deficient speech perception performance, thereby rendering them unable to complete the sentence in noise tests.

The Mandarin DINT was developed mainly employing methods recommended by the International Collegium of Rehabilitative Audiology (ICRA) for digit-in-noise test development for the assessment of speech understanding as an auditory function (Akeroyd et al., 2015). While previous DINTs employ 3 digits, the current study employed test stimuli consisted of 2 to 5 Mandarin digit sequences consisting of 10 monosyllabic digits (i.e., 0-2 and 4-10). The digit 3 is not used due to its need for a significantly greater adjustment (6.42 dB) to achieve similar intelligibility as other digits and being perceived as unnaturally loud by five Mandarin-speaking audiologists. A list of 24 digit sequences was created from a 120 (90 in 2-digit) fixed digit sequence corpus of each digit sequence in every test trial. The DINT was found to correlate well with speech perception in noise measured on the MHINT and CMNmatrix.

The MHINT was the first standardized Mandarin sentence perception in noise test utilizing the same development rationale as the other language HINTs. There are 12 20-sentence lists in the test corpus, with each sentence with ten characters. Between the lists, the distributions of phonemes and lexical tones are balanced. The sentences were written in a simple and conversational style that could be easily understood by people of various educational backgrounds (Wong et al., 2007).

The CMNmatrix test was established following ICRA guidelines, using the same paradigms as other language versions of the Matrix sentence test. Semantically unpredictable, syntactically fixed sentences are formed in the order of name-verb-number-adjective-object. All words are from a 50-word base matrix reflecting the distribution of Mandarin phonemes and lexical tones, with ten alternatives per category. Each test list contains 20 sentences. Compared to MHINT, the sentences in the CMNmatrix test contain less contextual information (Hu et al., 2018).

### Cognitive Function Tests

To screen for MCI among older participants while accounting for the low education level of older adults in China, the Chinese version of the Montreal Cognitive Assessment Basic (MoCA-BC) was used (Julayanont et al., 2015; Chen, 2016). The cut-off scores varied depending on the education level, with 19 for those with less than 6 years of education, 22 for 7 to 12 years of education, and 24 for over 12 years of education (Chen et al., 2016).

The Digit Span Test (DST) was used to measure the auditory working memory capacity, attention, and executive function, while the Corsi Block-Tapping Task (CBTT) was used to measure the visuospatial working memory capacity. Forward and backward DSTs used in this study were from the Chinese version of the Wechsler Adult Intelligence Scale (Gong, 1992). Participants were asked to listen and repeat a series of digit sequences recorded by a female speaker in forward and reverse orders. The number of digits ranged from 3 to 12 digits in the forward task and 2 to 10 in the backward task. The test was stopped when the participant failed to repeat the same length correctly twice. The test was scored as the sum of the longest number of digits that participants could repeat correctly in forward and backward tasks. Prior to the actual test, presentation level was adjusted to optimize speech clarity and clearest and comfortable for each participant.

The CBTT is a widely used test to assess visuospatial working memory capacity in clinical practice and research, somewhat similar to the DST (Kessels et al., 2000). The test used in the present study was provided by the Psy Toolkit (Stoet, 2010, 2017). In the test, the participants were told that a series of blocks of 9 blocks on the screen would light up in a random sequence. After the sound “go”, the participants were asked to use the mouse to click the blocks in the same forward or reverse orders. Participants unfamiliar with using the mouse could use their fingers to point at the target blocks displayed on a touch screen instead. The number of lighted blocks kept increasing until the participant failed to recall the same condition twice in forward and reverse order, respectively. The test was scored as the sum of the highest number of blocks in the forward and reverse order.

### Test Equipment

All hearing-related measurements were conducted in a sound-treated booth. An external monitor was used for conducting the CBTT. Sound signals were presented by a laptop connected to a TASCAM US 2×2 soundcard and JBL control 25-1 loudspeakers.

### Procedure

Otoscopy was performed to ensure the cleanliness and normalcy of the outer ear structures of participants. Pure tone audiometry was administered to measure hearing levels of participants. All participants were asked to wear their hearing aids during the following tests. The MoCA, DST, and CBTT were conducted sequentially. Before the formal DST, the Mandarin digits 1 to 9 used in the DST were played to the participants to familiarize them with the digits pronunciation. Subsequently, the DINT, MHINT, and CMNmatrix test were conducted sequentially in the sound field. There were six listening conditions: adaptive 2-, 3-, 4-, 5-digit DINT, one adaptive MHINT, and one open-set fixed 0 dB SNR. CMNmatrix sentence test were administered as practice before the actual test. The noise levels were set at 65 dBA, and test stimuli were presented in front of the participants at 0 degree azimuth, from a loudspeaker one meter away from the center of the head of participants. Participants were offered a break anytime they showed signs of fatigue or made a request.

### Results

### Cognitive Function Test

Twenty-one out of the 81 participants failed the MoCA-BC screening, indicating possible cognitive decline. All participants completed the DST and CBTT. Among all older participants, mean MoCA, DST, and CBTT scores were 22.14±3.37, 10.04±1.77, and 7.25±1.61, respectively. Shapiro-Wilk Normality Test was used to determine the normality of data distribution. Person correlation was used for normally distributed data, while Spearman's rank-order correlation was employed for non-normally distributed data. Results showed statistically significant negative correlations between age and all three cognitive tests (r=−0.382 to −0.257), as well as statistically significant, positive correlations between education and results from all three cognitive tests (r=0.492 to 0.595).

### Speech Perception in Noise Test Results

All participants completed the 2-digit, 3-digit, and 4-digit DINT, and two failed to complete the 5-digit DINT. Among the 81 older participants, 24 were unable to complete the MHINT and CMNmatrix test, and reported that the sentences were too fast and too long to understand and remember. Twelve participants neither passed the MoCA-BC screening nor were able to complete the two SIN tests. Among the 79 older participants who were able to complete all four digit sequences of the DINT, the SRTs of 2-, 3-, 4-, and 5-digit DINT were-1.30±3.47, −0.97±3.45, −0.67±3.48, and 0.94±4.30 dB SNR, respectively. Spearman's rank-order correlation revealed significant negative correlations between age (r=0.403 to 0.444, p<0.01) and hearing level (r=0.430 to 0.449, p<0.01) with all DINT digit sequences; education level was found to significantly correlate only with SRT measured on the 5-digit DINT (r=−0.285, p<0.05).

### The Speech Perception and Cognitive Function

Spearman's rank-order correlation was run to examine the relationship between DIN and three cognitive function tests. Significant correlations were observed between all DINT digit sequences and three cognitive function tests, with 5-digit DINT yielding the highest correlation coefficients. Pearson's partial correlation to correlate results from 5-digit DINT with cognitive function tests, while controlling for the effects of age and hearing level showed significant correlations were found with 5-digit DINT results only and not with other digit sequences.

A total of 79 participants who completed all digit sequences in the DINT were divided into two groups based on whether they passed the MoCA-BC.

An independent-samples t-test revealed statistically significant differences between participants who passed and failed MoCA-BC in 5-digit DINT, t(77)=−3.135, p=0.002.

Spearman's rank-order correlation revealed that the digit 5-2 and digit 5-3 had significant correlations with the MoCA-BC, DST, CBTT.

A one-way ANOVA revealed significant differences in SRT among the three groups: F(2, 110)=52.474, p<0.001, ω²=0.477, for digit 5-2 difference and for F(2, 110)=49.017, p<0.001, ω²=0.459, for digit 5-3 difference. Tukey post hoc analysis revealed significant differences in digit 5-2 and digit 5-3 differences between older participants who failed MoCA-BC and the two other groups (p<0.001). Digit 5-2 difference (p=0.331) and digit 5-3 difference (p=0.632) did not differ statistically between older participants who passed the MoCA-BC and young adults.

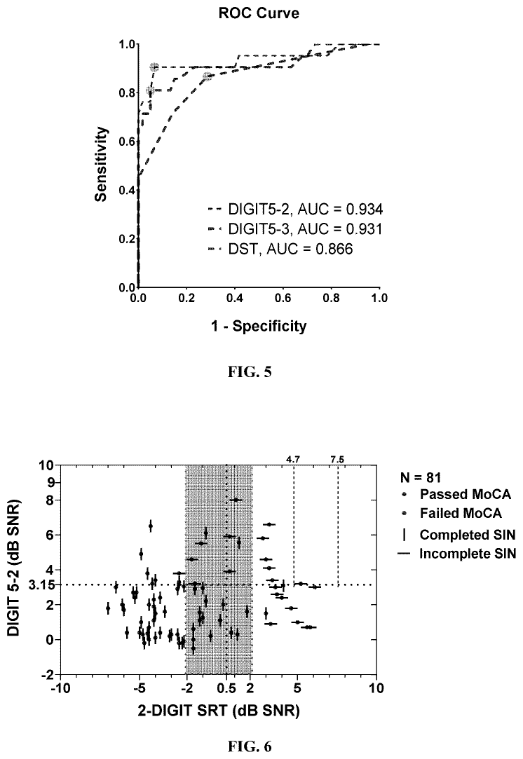

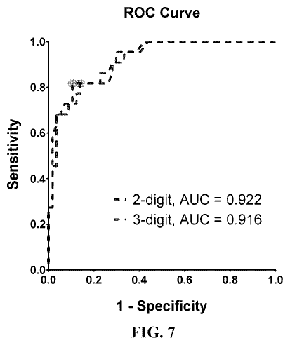

The effectiveness of using the DST, digit 5-3, digit 5-2 for general cognitive function screening were investigated, with participants classified as having MCI based on whether they passed MoCA-BC. Receiver operating characteristic (ROC) curves were employed to assess the ability of the DST, digit 5-2 and digit 5-3 to discriminate between participants with normal cognition or MCI. The area under the ROC curve (AUC) was calculated to compare the diagnostic performance of the DST, digit 5-2 and digit 5-3. The optimal cut-off scores for discriminating individuals with MCI were determined using the Youden index (J) method, which identifies the cut-off value that maximizes the Youden function, representing the difference between the true positive rate and the false positive rate across all potential cut-off values. High AUCs were found with the DST (0.866), digit 5-2 difference (0.934), and digit 5-3 difference (0.931) (see FIG. 5). The optimal cut-off score of the DST, digit 5-2 difference, and digit 5-3 difference were 8.5 (sensitivity: 0.867; specificity: 0.714), 3.15 (sensitivity: 0.905; specificity: 0.933), and 2.95 (sensitivity: 0.81; specificity: 0.905), respectively (see FIG. 6). Additionally, ROC curves were used to evaluate the ability of the 2-digit and 3-digit to discriminate between participants who were able to complete or failed to complete speech in noise test. High AUCs were found with 2-digit SRT (AUC=0.922) and 3-digit SRT (AUC=0.916) (see FIG. 7). The optimal cut-off score for 2-digit SRT, and 3-digit SRT were 0.5 (sensitivity: 0.818; specificity: 0.895), and 0.2 (sensitivity: 0.818; specificity: 0.860), respectively.

### Discussion

A Mandarin DINT was developed and validated. The time to complete a 2-digit, a 3-digit, a 4-digit, and a 5-digit DINT was approximately 1:30, 2:00, 2:25, and 2:55 minutes respectively. The following discusses the psychometric properties and the effects of the number of digits on SRT measurement.

### The SRT and Slope in the DINT

SRTs among both young and older adults increased as the number of digits increased. The mean SRTs for young adults increased by 1.09 dB, from −11.11 dB SNR using the 2-digit sequence to −10.02 dB SNR using the 5-digit sequence. The mean 3-digit SRT in young normal hearing adults (−10.99 dB SNR) in the current study was comparable to the mean 3-digit SRTs (−9.3 to −11.2 dB SNR) of the German, French, Dutch, Polish, Finnish and South African English versions (Jansen et al., 2010; Potgieter et al., 2016; Smits et al., 2013; Zokoll et al., 2012).

The slope steepened (from 16.58 to 21.09%/dB) as the number of digits increased from two to five. While the slope for the 3-digit sequence (18.79%/dB) agreed with the slopes of most language versions of the DTT (i.e., 15 to 20%/dB) (Van den Borre et al., 2021), it was slightly shallower than the slopes obtained in some other language versions, such as German (19.6%/dB), French (27.1%/dB) and South African English (20%/dB). As spectral information is efficiently masked in LTASS noise, languages that primarily rely on spectral information for speech recognition are expected to have steeper slopes compared to those (e.g., Chinese) relying on temporal information (Xu & Pfingst, 2008; Zokoll et al., 2012). It is not surprising, therefore, that a relatively shallow slope was obtained in the Mandarin DTT.

### Test-Retest Reliability and The Number of Digits

All digit sequences evaluated in the present study yielded good test-retest reliability. The measurement error equation used in this study removed possible training effects (Smits & Houtgast, 2005). In the present study, measurement error was about 0.3 dB in young adults and increased to about 0.7 dB in older adults with hearing loss, which compares well with the measurement errors of less than 1 dB obtained from the DTTs in most other languages (Van den Borre et al., 2021). Lower concentration and fatigue could result in increased measurement error among older adults (Koole et al., 2016). As different digit sequences were used for each test, the digit sequences might have interacted with the variations in hearing sensitivity across frequencies to affect the test-retest reliability among older adults, compared to young normal hearing adults. However, the difference was very small, and the measurement error was similar to when the digits had equal opportunities of appearance across lists in other language versions of the DTT (e.g., French version).

### Criterion Validity and The Number of Digits

The SRTs of all digit sequences correlated well with the SRTs measured using sentence-in-noise tests. Although the sentences in the CMNmatrix test contained less contextual information than those in the MHINT, no substantial differences were found between the SRTs obtained in the DINT and the SRTs obtained using the two sentence tests. These results compared favourably with those reported between the French DTT and the French Matrix Test (r=0.89) (Jansen et al., 2012) and between the Dutch DTT and Dutch sentence-in-noise test (r=0.87) (Smits et al., 2004).

### Working Memory Capacity and Number of Digits

Results from the two working memory capacity tests correlated with the 5-digit SRT but not with other digit sequences, regardless of whether age and hearing loss was controlled for. In other words, 2- to 4-digit sequences seemed to primarily reflect auditory perception, while the 5-digit sequence was also affected by working memory. According to the ELU model, a mismatch between speech input and phonological representations of sematic long-term memory occurs in speech perception around the SRT, and this mismatch is exacerbated when the speech input becomes complex. Working memory is then required to remedy the mismatch in order to achieve speech perception (Rönnberg et al., 2013, 2019, 2021). In the DINT, the increase in the number of digits increases the memory load, resulting in the need for more working memory resources to remedy the mismatch as the number increases. When individuals have sufficient and accessible working memory resources, the SRTs increase slowly with the increase in the number of digits in a sequence, as shown in our study, for young adults using 2-digit to 5-digit sequences and for older adults using 2-digit to 4-digit sequences, while the increase in SRT using the 5-digit sequence was more substantial in older adults.

### The Appropriate Number of Digits to Use in DINTs

The relationships between SRT and working memory capacity as demonstrated in the current study have important implications for determining the number of digits to use in a DINT. While 2- to 4-digit sequences reflect an individual's auditory perceptual function, working memory capacities have a greater impact on 5-digit SRTs, resulting in findings that should be interpreted differently. Although results from 2- to 4-digit sequences differed significantly, the difference was minimal (0.52 and 0.66 dB) among young normal hearing listeners and older adults with hearing loss. These digit sequences possess similar psychometric characteristics, good test-retest reliability and good criterion validity. They also correlated very highly with each other (rs≥0.97). Thus, SRT could be measured using any of these digit sequences. In other words, there is no evidence that suggests particular advantages in, or against, the use of 3-digit sequences, which have been used in most language versions of the DINT to evaluate speech understanding as an auditory function.

As mentioned earlier, other language versions of the DINT have been used successfully for hearing screening (Smits et al., 2013). Although the current study showed that the 2-digit sequence yielded the shallowest slope among the four digit sequences, the slope (16.58 dB/%) was still in the range of slopes of most DTTs (Van den Borre et al., 2021). Given that the 2-digit sequence yielded psychometric properties and SRTs similar to other digit sequences, and that less time is needed for SRT measurement, 2-digit sequences could be considered for hearing screening. On the other hand, the 5-digit SRT, with its relationship to working memory, can be used as an indicator for cognitive screening. Similarly, the results also showed that 5-2 and 5-3 digit differences are excellent measures to indicate the presence of cognitive decline.

### CONCLUSION

This study developed the Mandarin DINT using four digit sequences and showed that it had good test-retest reliability and criterion validity, and provided evidence for the first time concerning the selection of digit sequence. While the results provide support for the use of a 3-digit sequence, as in the case of other language versions of the DTT, 2-digit sequences could yield similar results in a shorter time and be used for hearing screening. Given the relationship with cognitive function tests, results from the 5-digit sequence and the difference in SRT measured using shorter digit sequences compared to 5 digit sequences could be used to indicate the need for referral for cognitive function evaluation.

Although the experiments were performed on the Mandarin language, the same principles are applicable to DINT in other languages. Thus, the disclosed methods can be used to identify speech understanding difficulties associated with hearing loss and additional impact from cognitive decline, any language of interest, for example, English, Mandarin, Cantonese, French, German, Spanish, Japanese, Portuguese, etc., The experiments described here could be replicated with DINT in other languages to establish the cutoff points for cognitive function referral. In other words, the concepts established in the current application are governed by the nature of auditory perception and cognitive function, thus are universal and applicable to other languages.

### REFERENCES

Akeroyd, M. A. (2008). Are individual differences in speech reception related to individual differences in cognitive ability? A survey of twenty experimental studies with normal and hearing-impaired adults. Int. J. Audiol., 47(SUPPL. 2). https://doi.org/10.1080/14992020802301142
Akeroyd, M. A., Arlinger, S., Bentler, R. A., Boothroyd, A., Dillier, N., Dreschler, W. A., Gagné, J.-P., Lutman, M., Wouters, J., Wong, L., & Kollmeier, B. (2015). International Collegium of Rehabilitative Audiology (ICRA) recommendations for the construction of multilingual speech tests. Int. J. Audiol., 54(sup2), 17-22. https://doi.org/10.3109/14992027.2015.1030513
Cullington, H. E., & Aidi, T. (2017). Is the digit triplet test an effective and acceptable way to assess speech recognition in adults using cochlear implants Implants Int., 18(2), 97-105. in a home environment? Cochlear https://doi.org/10.1080/14670100.2016.1273435
Gong, Y. X. (1992). Manual of Wechsler Adult Intelligence Scale-Chinese Version. Chinese Map Press.
Hecker, K., & Violato, C. (2009). Validity, Reliability, and Defensibility of Assessments in Veterinary Education. J. Vet. Med. Educ., 36(3), 271-275. https://doi.org/10.3138/jvme.36.3.271
Holube, I., Fredelake, S., Vlaming, M., & Kollmeier, B. (2010). Development and analysis of an International Speech Test Signal (ISTS). Int. J. Audiol., 49(12), 891-903. https://doi.org/10.3109/14992027.2010.506889
Hu, H., Xi, X., Wong, L. L. N., Hochmuth, S., Warzybok, A., & Kollmeier, B. (2018). Construction and evaluation of the Mandarin Chinese matrix (CMNmatrix) sentence test for the assessment of speech recognition in noise. Int. J. Audiol., 57(11), 838-850. https://doi.org/10.1080/14992027.2018.1483083
Jansen, S., Luts, H., Wagener, K. C., Frachet, B., & Wouters, J. (2010). The French digit triplet test: A hearing screening tool for speech intelligibility in noise. Int. J. Audiol., 49(5), 378-387. https://doi.org/10.3109/14992020903431272
Jansen, S., Luts, H., Wagener, K. C., Kollmeier, B., Del Rio, M., Dauman, R., James, C., Fraysse, B., Vormès, E., Frachet, B., Wouters, J., & van Wieringen, A. (2012). Comparison of three types of French speech-in-noise tests: A multi-center study. Int. J. Audiol., 51(3), 164-173. https://doi.org/10.3109/14992027.2011.633568
Kaandorp, M. W., Smits, C., Merkus, P., Goverts, S. T., & Festen, J. M. (2015). Assessing speech recognition abilities with digits in noise in cochlear implant and hearing aid users. Int. J. Audiol., 54(1), 48-57. https://doi.org/10.3109/14992027.2014.945623
Koole, A., Nagtegaal, A. P., Homans, N. C., Hofman, A., Baatenburg de Jong, R. J., & Goedegebure, A. (2016). Using the Digits-In-Noise Test to Estimate Age-Related Hearing Loss. Ear Hear., 37(5), 508-513. https://doi.org/10.1097/AUD.0000000000000282
Kwak, C., Seo, J.-H., Oh, Y., & Han, W. (2022). Efficacy of the Digit-in-Noise Test: A Systematic Review and Meta-Analysis. J. Audiol. Otol., 26(1), 10-21. https://doi.org/10.7874/jao.2021.00416
Lyzenga, J., & Smits, C. (2011). Effects of Coarticulation, Prosody, and Noise Freshness on the Intelligibility of Digit Triplets in Noise. J. Am. Acad. Audiol., 22(04), 215-221. https://doi.org/10.3766/jaaa.22.4.4
Nyberg, L., Lövden, M., Riklund, K., Lindenberger, U., & Bäckman, L. (2012). Memory aging and brain maintenance. Trends Cogn. Sci., 16(5), 292-305. https://doi.org/10.1016/j.tics.2012.04.005
Potgieter, J.-M., Swanepoel, D. W., Myburgh, H. C., Hopper, T. C., & Smits, C. (2016). Development and validation of a smartphone-based digits-in-noise hearing test in South African English. Int. J. Audiol., 55(7), 405-411. https://doi.org/10.3109/14992027.2016.1172269
Potgieter, Swanepoel, D. W., & Smits, C. (2018). Evaluating a smartphone digits-in-noise test as part of the audiometric test battery. South African J. Commun. Disord.=Die Suid-Afrikaanse Tydskr. Vir Kommun., 65(1), e1-e6. https://doi.org/10.4102/sajcd.v65i1.574
Rönnberg, J., Holmer, E., & Rudner, M. (2019). Cognitive hearing science and ease of language understanding. Int. J. Audiol., 58(5), 247-261. https://doi.org/10.1080/14992027.2018.1551631
Rönnberg, J., Holmer, E., & Rudner, M. (2021). Cognitive Hearing Science: Three Memory Systems, Two Approaches, and the Ease of Language Understanding Model. J. Speech, Lang. Hear. Res., 64(2), 359-370. https://doi.org/10.1044/2020_JSLHR-20-00007
Rönnberg, J., Lunner, T., Zekveld, A., Sörqvist, P., Danielsson, H., Lyxell, B., Dahlström, Ö., Signoret, C., Stenfelt, S., Pichora-Fuller, M. K., & Rudner, M. (2013). The Ease of Language Understanding (ELU) model: theoretical, empirical, and clinical advances. Front. Syst. Neurosci., 7 (JUNE), 1-17. https://doi.org/10.3389/fnsys.2013.00031
Smits, C., & Houtgast, T. (2005). Results From the Dutch Speech-in-Noise Screening Test by Telephone. Ear Hear., 26(1), 89-95. https://doi.org/10.1097/00003446-200502000-00008
Smits, C., & Houtgast, T. (2007). Recognition of digits in different types of noise by normal-hearing and hearing-impaired listeners. Int. J. Audiol., 46(3), 134-144. https://doi.org/10.1080/14992020601102170
Smits, C., Kapteyn, T. S., & Houtgast, T. (2004). Development and validation of an automatic speech-in-noise screening test by telephone. Int. J. Audiol., 43(1), 15-28. https://doi.org/10.1080/14992020400050004
Smits, C., Theo Goverts, S., & Festen, J. M. (2013). The digits-in-noise test: Assessing auditory speech recognition abilities in noise. J. Acoust. Soc. Am., 133(3), 1693-1706. https://doi.org/10.1121/1.4789933
Souza, P., & Arehart, K. (2015). Robust relationship between reading span and speech recognition in noise. Int. J. Audiol., 54(10), 705-713. https://doi.org/10.3109/14992027.2015.1043062
Tripathi, R., Kumar, K., Bharath, S., P, M., Rawat, V. S., & Varghese, M. (2019). Indian older adults and the digit span A preliminary report. Dement. Neuropsychol., 13(1), 111-115. https://doi.org/10.1590/1980-57642018dn13-010013
Van den Borre, E., Denys, S., van Wieringen, A., & Wouters, J. (2021). The digit triplet test: a scoping review. Int. J. Audiol., 60(12), 946-963. https://doi.org/10.1080/14992027.2021.1902579
Versfeld, N. J., Daalder, L., Festen, J. M., & Houtgast, T. (2000). Method for the selection of sentence materials for efficient measurement of the speech reception threshold. J. Acoust. Soc. Am., 107(3), 1671-1684. https://doi.org/10.1121/1.428451
Wilson, R. H., Burks, C. A., & Weakley, D. G. (2005). A comparison of word-recognition abilities assessed with digit pairs and digit triplets in multitalker babble. J. Rehabil. Res. Dev., 42(4), 499. https://doi.org/10.1682/JRRD.2004.10.0134
Wong, L. L. N., Soli, S. D., Liu, S., Han, N., & Huang, M.-W. (2007). Development of the Mandarin Hearing in Noise Test (MHINT). Ear Hear., 28(2), 70S-74S. https://doi.org/10.1097/AUD.0b013e31803154d0
Wong, Yu, J. K. Y., Chan, S. S., & Tong, M. C. F. (2014). Screening of Cognitive Function and Hearing Impairment in Older Adults: A Preliminary Study. Biomed Res. Int., 2014, 1-7. https://doi.org/10.1155/2014/867852
Xu, L., & Pfingst, B. E. (2008). Spectral and temporal cues for speech recognition: Implications for auditory prostheses. Hear. Res., 242(1-2), 132-140. https://doi.org/10.1016/j.heares.2007.12.010
Zokoll, M. A., Wagener, K. C., Brand, T., Buschermöhle, M., & Kollmeier, B. (2012). Internationally comparable screening tests for listening in noise in several European languages: The German digit triplet test as an optimization prototype. Int. J. Audiol., 51(9), 697-707. https://doi.org/10.3109/14992027.2012.690078

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain, using no more than routine experimentation, many equivalents to the specific embodiments of the method and compositions described herein. Such equivalents are intended to be encompassed by the following claims.

1. A method of testing a participant's ability to accurately detect speech in the presence of background noise includes: (a) audibly presenting a series of 2 digits, 3 digits, 4 digits and/or 5 digits, in random or fixed orders, to a first ear of a participant in quiet or in noise at a fixed or varied signal-to-noise ratios (SNRs), (b) audibly presenting a series of 2 digits, 3 digits, 4 digits and/or 5 digits, in random or fixed orders, to a second ear of a participant in quiet or in noise at a fixed or random signal-to-noise ratios (SNRs), wherein the digits are presented in a language native to the participant or in which the participant is fluent, (c) recording digits or digit sequences identified correctly by the participant and (d) calculating scores based on the percentage of digits or digit sequences repeated correctly by the participant or the difference in level between speech and noise to yield 50% of the digit sequences being identified correctly.

2. The method of claim 1, wherein the series of digits are audibly presented from a laptop connected to a soundcard and loudspeakers, a smartphone or tablet connected to loudspeakers or headphones and calibrated to deliver the signals at appropriate levels.

3. The method of claim 1, further comprising evaluating the ability to repeat shorter digit sequences in forward order including 2 or 3 digits, and comparing speech recognition using forward shorter digit sequence versus a forward longer digit sequence of 4 or 5 digits.

4. The method of claim 1, further comprising comparing speech recognition using a forward shorter digit sequence versus a backward shorter digit sequence of 2 or 3 digits.

5. The method of claim 1, comprising comparing speech recognition using forward shorter digit sequence versus forward shorter digit sequence with shorter intervals between digits, including forward 2 or 3 digits with 200 ms intervals versus forward 2 or 3 digits.

6. The method of claim 1, wherein the language is selected from the group consisting of English, Mandarin, Cantonese, French, German, Spanish, Japanese and Portuguese etc.

7. The method of claim 1, wherein the digits and noise are audibly presented to the participants in each ear individually or two ears simultaneously.

8. The method of claim 1, wherein recording digits or digit sequences correctly identified by the participant comprises asking the participant to repeat the digits heard or identify the digits by taping the word as seen on a computer keyboard or screen, by any user.

9. The method of claim 1, wherein results obtained using shorter digit sequences are compared to those obtained using longer digit sequences, including 5-2 or 5-3 difference, and differences compared to a standard value or cut-of score to identify the presence of a cognitive decline, wherein the standard or cut-off score is determined by testing a group of participants who are speaking the language as native speakers and have no known cognitive decline or determined by measuring reaction time.

10. An apparatus for measuring digit recognition in relation to hearing loss, comprising:
a speaker measuring device, comprising:
a first pairing connection module;
a first display configured to show a sequence of characters or numbers serving as test data; and
a first signal transmitting module configured to transmit the test data via the first pairing connection module; and
a listener measuring device, comprising:
a second pairing connection module configured to electrically communicate with the first pairing connection module;
a second display configured to show an indicator signal and display a virtual keypad;
a receiver configured to receive the test data from the speaker measuring device;
an audio recorder configured to record environmental sound data;
a testing recorder configured to record a test response inputted from the virtual keypad; and
a scoring module configured to compare the test response with the test data to generate score data.

11. The apparatus of claim 10, wherein the listener measuring device further comprises a second signal transmitting module configured to package the score data and the environmental sound data and transmit them via wireless communication.

12. The apparatus of claim 11, further comprising a hearing device electrically communicating with the listener measuring device, wherein the second signal transmitting module is further configured to signal the hearing device to adjust signal level of the hearing device up when the scoring module indicates a signal for a wrongly repeating condition.

13. The apparatus of claim 12, wherein the second signal transmitting module is further configured to signal the hearing device to adjust signal level of the hearing device down when the scoring module indicates a signal for a correct repeating condition, and the adjustment to the signal levels including adjusting up and down is recorded in the listener measuring device as a threshold for digit recognition of the test data.

14. The apparatus of claim 10, wherein the score data includes a correct percentage for the test response relative to the test data.

15. A method for measuring digit recognition in relation to hearing loss, comprising:
pairing a speaker measuring device and a listener measuring device by using a first pairing connection module of the speaker measuring device and a second pairing connection module of the listener measuring device;
showing, by a first display of the speaker measuring device, a sequence of characters or numbers serving as test data;
transmitting, by a first signal transmitting module of the speaker measuring device, the test data to the listener measuring device via the first pairing connection module;
showing, by a second display of the listener measuring device, an indicator signal and display a virtual keypad;
receiving, by a receiver of the listener measuring device, the test data from the speaker measuring device;
recording, by an audio recorder of the listener measuring device, environmental sound data;
recording, by a testing recorder of the listener measuring device, a test response inputted from the virtual keypad; and
comparing the test response with the test data to generate score data.

16. The method of claim 15, further comprising:
packaging, by a second signal transmitting module of the listener measuring device, the score data and the environmental sound data and transmitting them via wireless communication.

17. The method of claim 16, further comprising:
electrically communicating the listener measuring device with a hearing device; and
signaling, by the second signal transmitting module, the hearing device to adjust signal level of the hearing device up when a scoring module indicates a signal for a wrongly repeating condition.

18. The method of claim 17, further comprising:
signaling, by the second signal transmitting module, the hearing device to adjust signal level of the hearing device down when the scoring module indicates a signal for a correct repeating condition, wherein the adjustment to the signal levels including adjusting up and down is recorded in the listener measuring device as a threshold for digit recognition of the test data.

19. The method of claim 15, wherein the score data includes a correct percentage for the test response relative to the test data.

20. A computer-implement method (CIM) for assessing a participant's ability to detect speech, the CIM comprising:
(i) audibly presenting one or more digit sequences containing two or more digits, in random or fixed orders, to the participant,
(ii) scoring how many digit sequences the participant correctly identifies, and
(iii) displaying a result on the assessment on a graphical user interface, optionally with information to interpret the result,
wherein, digits in a digit sequence are homogenous in difficulty and/or homogenous in psychometric functions.

21. The CIM of claim 20, wherein the two or more digits in step (i) are obtained or processed from one or more individuals speaking the same native language.

22. The CIM of claim 20, wherein in step (ii), the participant identifies a digit sequence presented by repeating a digit sequence aloud or inputting a response using a keypad.

23. The CIM of claim 20, wherein assessing the participant's ability to detect speech is performed via an adaptive speech recognition threshold (SRT) mode or a fixed signal-to-noise ratio (SNR) mode.

24. A computer-implemented method (CIM) for developing test materials for assessing a participant's ability to detect speech, the CIM comprising:
(a1) processing digits to be included in the test materials to introduce homogeneity in difficulty, psychometric functions, or a combination thereof, wherein the digits are processed on a processing hardware platform.

25. The CIM of claim 24, wherein the digits are obtained from one or more individuals (b1) speaking the same native language, (b2) from the same ethnic group, and/or (b3) from the same geographical region as the participant.

26. The CIM of claim 24, wherein processing the digits further involve generating a deep learning artificial intelligence based model from another set of digits data, to provide predicted adjustment levels for each digit.
